(19) United States
(12) Patent Application Publication
(10) Pub. No.: US 2012/0065199 A1
(43) Pub. Date: Mar. 15, 2012

(54) SUBSTITUTED QUINOLINES FOR USE AS VEGF INHIBITORS

(75) Inventors: Johan Malm, Trangsund (SE); Rune Ringom, Uppsala (SE); Patrizia Caldirola, Uppsala (SE); Jacob Westman, Jarlasa (SE)

(73) Assignee: CLANOTECH AB, Solna (SE)

(21) Appl. No.: 13/321,602

(22) PCT Filed: May 20, 2010
(86) PCT No.: PCT/EP10/56968

§ 371 Date: Nov. 21, 2011

(30) Foreign Application Priority Data
May 20, 2009 (EP) 09160829.9

(51) Int. Cl. A61K 31/5377; C07D 413/12; C07D 401/12; A61P 9/10; C07D 405/12; A61P 35/00; A61P 27/02; A61P 29/00; A61K 31/4709; A61P 17/06

(52) U.S. Cl. 514/232.8; 514/313; 544/128; 546/159

(57) ABSTRACT

A compound of formula (I)

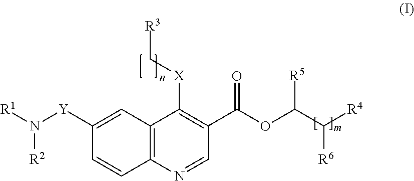

as well as pharmaceutically acceptable salts thereof and pharmaceutical compositions including a therapeutically effective amount of the compounds. The compound is useful in treatment of cancer, diabetic retinopathy, age-related macular degeneration, inflammation, stroke, ischemic myocardium, atherosclerosis, macular edema and psoriasis.

### FIELD OF THE INVENTION

The present invention relates to substituted carboxylic acid esters of 3-carboxylic quinoline derivatives and to the use thereof in therapy. These esters display improved uptake in vivo and are hydrolyzed to their corresponding carboxylic acids in vivo. Particularly, the present invention relates to quinoline derivatives for the treatment of cancer, diabetic retinopathy, age-related macular degeneration, inflammation, stroke, ischemic myocardium, atherosclerosis, macular edema and psoriasis.

### BACKGROUND OF THE INVENTION

The following review of the state of art is only provided to aid the understanding of the present invention and neither it nor any of the references cited within it are admitted to be prior art to the present invention.

Angiogenesis, the outgrowth of new capillaries from pre-existing vessels, is essential for embryonic development, organ formation, tissue regeneration, and remodeling [Folkman, J. & Shing, Y. (1992) J. Biol. Chem. 267, 10931-10934]. It also contributes to the development and progression of a variety of pathological conditions, including tumor growth and metastasis, cardiovascular diseases, diabetic retinopathy, rheumatoid arthritis, psoriasis [Folkman, J. Nat. Med. 1995, 1, 27-30] and age-related macular degeneration [Barakat, M. R.; Kaiser, P. K. Expert Opin. Investig. Drugs 2009, 18, 637-46; Chappelow, A. V.; Kaiser, P. K. Drugs 2008, 68, 1029-1036].

Angiogenesis and vasculogenesis are complex multistep processes that include proliferation, migration and differentiation of endothelial cells, degradation of the extracellular matrix, tube formation, and sprouting of new capillary branches [Hanahan, D.; Folkman, J. Cell 1996, 86, 353-364; Risau, W. Nature (London) 1997, 386, 671-674]. The complexity of the angiogenic processes suggests the existence of multiple controls of the system, which can be transiently switched on and off. A switch of the angiogenic phenotype in tissues is thought to depend on a local change of the balance between angiogenic stimulators and inhibitors [Folkman, J. N. Engl. J. Med. 1995, 333, 1757-1763].

Among many described angiogenic factors, vascular endothelial growth factor (VEGF)/vascular permeability factor is one of the best-characterized positive regulators with its distinct specificity for vascular endothelial cells [Senger, D. R.; Galli, S. J.; Dvorak, A. M.; Perruzzi, C. A.; Harvey, V. S.; Dvorak, H. F. Science 1983, 219, 983-985; Ferrara, N.; Henzel, W. J. Biochem. Biophys. Res. Commun. 1989, 161, 851-858; Gospodarowicz, D.; Abraham, J. A.; Schilling, J. Proc. Natl. Acad. Sci. USA 1989, 86, 7311-7315]. The biological actions of VEGF include stimulation of endothelial cell proliferation, migration, differentiation, tube formation, increase of vascular permeability, and maintenance of vascular integrity [Mustonen, T.; Alitalo, K. J. Cell Biol. 1995, 129, 895-898; Ferrara, N.; Davis-Smyth, T. Endocr. Rev. 1997, 18, 4-25; Thomas, K. J. Biol. Chem. 1996, 271, 603-606; Risau, W. Nature (London) 1997, 386, 671-674; Breier, G.; Risau, W. Trends Cell Biol. 1997, 6, 451 156]. The angiogenic responses induced by VEGF are mediated by tyrosine kinase receptors, which are expressed primarily on vascular cells of the endothelial lineage [Mustonen, T.; Alitalo, K. J. Cell Biol. 1995, 129, 895-898; De Vries, C.; Escobedo, J. A.; Ueno, H.; Huck, K.; Ferrara, N.; Williams, L. T. Science 1992, 255, 989-99; Terman, B. I.; Dougher-Vermazen, M.; Carrion, M. E.; Dimitrov, D.; Armellino, D. C.; Gospodorawicz, D.; Bohlen, P. Biochem. Biophys. Res. Commun. 1992, 187, 1579-1586].

Inhibition of cell adhesion to the endothelial cell membrane (ECM), the fundamental step for activation, survival, targeting and migration of activated endothelial cells, might be one of the most promising target mechanisms for anti-angiogenesis. Not only VEGF is involved in these mechanisms but many of these interactions are also mediated by integrins, a family of multifunction cell adhesion receptors [Stupack, D. G. Oncology (Williston Park) 2007, 21 (9 Suppl 3), 6-12; Avraamides, C. J.; Garmy-Susini, B.; Varner, J. A. Nat. Rev. Cancer 2008, 8, 604-17.]. Members of the integrin family are non-covalent alpha/beta heterodimers that mediate cell-cell, cell-extracellular matrix and cell-pathogene interactions. They are also are believed to modulate the effect of receptors for vascular endothelial growth factor (VEGFRs) [Napione, L.; Cascone, I.; Mitola, S.; Serini, G.; Bussolino, F. Autoimmun. Rev. 2007, 7, 18-22].

Until now, 19 different integrin alpha subunits and 8 different beta subunits are known that combine to form at least 24 different alpha/beta heterodimers with different ligand specificity [Silva, R.; D'Amico, G.; Hodivala-Dilke, K. M.; Reynolds, L. E. Arterioscler Thromb Vasc Biol, 2008, 28, 1701-1713]. Of the presently approximately 24 known integrins, 16 have been reported to have involvement in some aspects of vascular biology. Of these α1β1, α2β1, α3β1, α5β1, α6β1, α6β4, αvβ3, and αvβ5 are known to be present in endothelial cells [Rupp, P. A.; Little, C. D. Circ. Res., 2001, 566-572; Stupack, D. G.; Cheresh, D. A. Sci. STKE, 2002, PE7], while vascular smooth muscle cells have been reported to have α1β1, α2β1, α3β1, α4β1, α5β1, α6β1, α7β1, α8β1, α9β1, αvβ1, αvβ3, αvβ5, and α6β4 [Moiseeva, E. P. Cardivasc. Res., 2001, 372-386].

The ligands for the extracellular domain of many integrins are the proteins of the extracellular matrix and the intracellular domain of the integrins are either directly or indirectly connected to intracellular components such as kinases and the cytoskeleton. Integrins serve as bidirectional signalling receptors, whereby protein activities and gene expression are changed by integrins in response to ligand binding to the extracellular domain thereof, which is also referred to as outside-in-signalling. On the other hand, the affinity of the integrins is modulated in response to intracellular changes such as binding of proteins to the extracellular domain of the integrin, which is referred to as inside-out signalling [Humphries, M. J. Biochem. Soc. Trans. 2000, 28, 311-339; Hynes, R. O. Cell, 2002, 110, 673-687].

Several studies on the integrin pattern on activated endothelial cells, mice gene knockouts and inhibition studies in angiogenic animal models with antibodies, peptides and small molecules have provided information about integrins and ECM proteins involved in critical steps of angiogenesis [Brooks, P. C.; Clark, R. A.; Cheresh, D. A. Science, 1994, 264, 569-571; Brooks, P. C. Eur. J. Cancer, 1996, 32A, 2423-2429; Mousa, S. A. Curr Opin Chem Biol, 2002, 6, 534-541; Hynes, R. O. Nature Medicine 2002, 8, 918-21; Kim, S.; Bell, K.; Mousa, S. A.; Varner, J. A.; Am. J. Pathol. 2000, 156, 1345-1362].

From studies referred to herein above it appeared that the vitronectin receptors αvβ3, αvβ5 and the fibronectin receptor α5β1 play a critical role in angiogenesis. Integrin α5β1 expression is significantly upregulated in blood vessels in human tumors and after stimulation with growth factors and, once expressed, α5β1 regulates the survival and migration of endothelial cells in vitro and in vivo. Integrin α5β1 is poorly expressed on quiescent endothelium but its expression is significantly upregulated on endothelium during tumor angiogenesis in both mice and humans, which make α5β1 a viable target for anti-angiogenic therapy [Kim, S.; Bell, K.; Mousa, S. A.; Varner, J. A.; Am. J. Pathol. 2000, 156, 1345-1362; Bhaskar, V.; Zhang, D.; Fox, M.; Seto, P.; Wong, M. H.; Wales, P. E.; Powers, D.; Chao, D. T; Dubridge, R. B.; Ramakrishnan, V. J. Transl. Med. 2007, 27, 61]. Expression of this integrin is also upregulated during corneal angiogenesis [Muether, P. S.; Dell, S.; Kociok, N.; Zahn, G.; Stragies, R.; Vossmeyer, D.; Joussen, A. M.; Exp. Eye. Res. 2007, 85, 356-365].

Combination of anti-angiogenetic therapy and other therapeutic approaches, such as chemotherapy, radiotherapy and gene therapy has also been applied and suggested for cancer treatment. Mounting evidence suggests that there is potentially synergistic effect of combined therapeutic approaches over single modality alone [Huveneers, S.; Truong, H.; Danen, H. J. Int. J. Radiat. Biol. 2007, 83, 743-751; Huber, P. E.; Bischof, M.; Jenne, J.; Heiland, S.; Peschke, P.; Saffrich, R.; Gröne, H. J.; Debus, J.; Lipson, K. E.; Abdollahi, A. Cancer Res. 2005, 65, 3643-3655].

WO 2008/119771 discloses C₁-C₆ alkyl esters of quinoline-3-carboxylic acid derivatives acting as tyrosine kinase inhibitors for treatment and prevention of cell proliferative disorders or cell differentiation disorders associated with abnormal tyrosine kinase activities.

### SUMMARY OF THE INVENTION

The present inventors now have found that novel quinoline derivatives with certain side-chain pattern are capable of efficiently blocking tumor growth in a mammal. Compared to similar analogs in the field, the compounds of the present invention also have improved solubility properties and improved in vitro properties.

Consequently, according to one aspect, the present invention relates to a compound of formula (I)

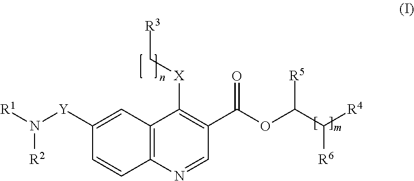

wherein

n is 0 (zero) or 1;

m is 0 (zero), 1 or 2;

R¹ and R² are independently selected from hydrogen; branched or unbranched C₁-C₈ alkyl, C₂-C₈ alkenyl or C₂-C₈ alkynyl; monocyclic or bicyclic, saturated or unsaturated C₃-C₈ carbocyclyl; and monocyclic or bicyclic, saturated or unsaturated C₁-C₇ heterocyclyl wherein each heteroatom is independently selected from N, O and S; said alkyl, alkenyl, alkynyl, carbocyclyl or heterocyclyl optionally being substituted with 1, 2 or 3 groups R^{a};

R³ is selected from monocyclic or bicyclic C₆-C₁₀ aryl; and monocyclic or bicyclic C₁-C₉ heteroaryl or heterocyclyl, wherein in said heteroaryl and heterocyclyl each heteroatom is independently selected from N, O and S; said aryl, heteroaryl or heterocyclyl optionally being substituted with 1, 2, 3, 4 or 5 groups R^{b};

R⁴ is selected from —OC(O)R⁷; —C(O)OR⁷; —NR⁷R⁸; —C(O)NR⁷R⁸; monocyclic or bicyclic C₁-C₉ heteroaryl; and monocyclic or bicyclic, saturated or unsaturated C₁-C₉ heterocyclyl, wherein said heteroaryl and heterocyclyl optionally contain an oxo group in the ring, and wherein in said heteroaryl and heterocyclyl each heteroatom independently is selected from N, O and S; said heteroaryl and heterocyclyl optionally being substituted with 1, 2 or 3 groups R^{a};

R⁵ and R⁶ are independently selected from hydrogen; and branched or unbranched C₁-C₄ alkyl, C₂-C₄ alkenyl or C₂-C₄ alkynyl; said alkyl, alkenyl and alkynyl optionally being substituted with 1, 2, or 3 groups independently selected from fluorine and chlorine;

R⁷ is selected from hydrogen; branched or unbranched C₁-C₄ alkyl, C₂-C₄ alkenyl or C₂-C₄ alkynyl; and phenyl; said alkyl, alkenyl, alkynyl and phenyl optionally being substituted with 1, 2, or 3 groups independently selected from fluorine and chlorine;

R⁸ is selected from hydrogen; branched or unbranched C₁-C₄ alkyl, C₂-C₄ alkenyl or C₂-C₄ alkynyl; monocyclic or bicyclic C₆-C₁₀ aryl; —S(O)₂R⁹; —C(O)OR⁹; and —C(O)R¹⁰; said alkyl, alkenyl, alkynyl or aryl optionally being substituted with 1, 2, or 3 halogen(s);

R⁹ is selected from hydrogen and branched or unbranched C₁-C₄ alkyl, C₂-C₄ alkenyl or C₂-C₄ alkynyl; said alkyl, alkenyl and alkynyl optionally being substituted with 1, 2, or 3 groups independently selected from fluorine and chlorine;

R¹⁰ is selected from hydrogen; branched or unbranched C₁-C₄ alkyl, C₂-C₄ alkenyl or C₂-C₄ alkynyl; and C₆ aryl; said aryl optionally being substituted with 1, 2 or 3 groups R^{a}; and said alkyl, alkenyl and alkynyl optionally being substituted with 1, 2, or 3 groups independently selected from fluorine and chlorine;

Y is selected from —C(O)—; —S(O)—; and —S(O)₂—;

X is selected from —NR^{c}—; —O—; and —S—;

each R^{a} is independently selected from halogen; hydroxy; carbonyl; methoxy; halomethoxy; dihalomethoxy; and trihalomethoxy;

each R^{b} is independently selected from halogen; carboxy; hydroxy; cyano; C₁-C₄ alkyl; C₂-C₄ alkenyl; C₂-C₄ alkynyl; C₁-C₄ alkyloxy; C₂-C₄ alkenyloxy; C₂-C₄ alkynyloxy; C₁-C₄ alkylthio; C₂-C₄ alkenylthio; C₂-C₄ alkynylthio; C₁-C₄ alkyl, C₂-C₄ alkenyl or C₂-C₄ alkynyl secondary or tertiary amino; C₁-C₄ alkyl, C₂-C₄ alkenyl or C₂-C₄ alkynyl secondary or tertiary amido; C₁-C₄ alkyl, C₂-C₄ alkenyl or C₂-C₄ alkynyl carbonyl; C₁-C₄ alkyl, C₂-C₄ alkenyl or C₂-C₄ alkynyl sulfonyl; C₁-C₄ alkyl, C₂-C₄ alkenyl or C₂-C₄ alkynyl sulfonyloxy; C₁-C₄ alkyl, C₂-C₄ alkenyl or C₂-C₄ alkynyl secondary or tertiary sulphonamido; C₁-C₄ alkyl, C₂-C₄ alkenyl or C₂-C₄ alkynyl silyl; and C₁-C₄ alkyloxy, C₂-C₄ alkenyloxy, or C₂-C₄ alkynyloxy carbonyl; wherein any alkyl, alkenyl and alkynyl moiety optionally is substituted with 1, 2 or 3 groups independently selected from halogen, hydroxy, methoxy, halomethoxy, dihalomethoxy and trihalomethoxy; and

R^{c} is selected from hydrogen; and branched or unbranched C₁-C₄ alkyl, C₂-C₄ alkenyl or C₂-C₄ alkynyl;

wherein any Cₚ alkyl, alkynyl and alkenyl group having a number p≧4 of carbon atoms optionally includes a C_{q} carbocyclic portion of q of carbon atoms, whereby 3≦q<p;

or a pharmaceutically acceptable salt thereof.

Another aspect of the invention relates to a compound of formula (I) as defined herein above, or a pharmaceutically acceptable salt thereof, for use in therapy.

Another aspect of the invention relates to a pharmaceutical composition comprising a therapeutically effective amount of a compound of formula (I) as defined herein above, or a pharmaceutically acceptable salt thereof, together with at least one pharmaceutically acceptable excipient. In one embodiment of this aspect said pharmaceutical composition comprises at least one further, pharmaceutically active compound. Said further pharmaceutically active compound may have anti-tumor activity.

Another aspect of the invention provides compounds of formula (I) or pharmaceutically acceptable salts thereof, for use in the treatment of diseases or disorders such as cancer, diabetic retinopathy, age-related macular degeneration, inflammation, stroke, ischemic myocardium, atherosclerosis, macular edema and psoriasis.

Another aspect of the invention provides the use of the compounds of formula (I) or pharmaceutically acceptable salts thereof, in the manufacture of a medicament for the treatment of disorders such as cancer, diabetic retinopathy, age-related macular degeneration, inflammation, stroke, ischemic myocardium, atherosclerosis, macular edema and psoriasis.

Another aspect of the invention provides a method of treating a mammal suffering from cancer, diabetic retinopathy, age-related macular degeneration, inflammation, stroke, ischemic myocardium, atherosclerosis, macular edema or psoriasis, comprising administering to said mammal in need thereof, a therapeutically effective amount of a compound of formula (I) or a pharmaceutically acceptable salt thereof. In one embodiment of this aspect, said mammal is a human.

Another aspect of the invention provides a method of treating a mammal suffering from a disease or disorder related to VEGFR tyrosine kinase or integrin activity, comprising administering to said mammal in need thereof, a therapeutically effective amount of a compound of formula (I) or a pharmaceutically acceptable salt thereof. In one embodiment of this aspect, said mammal is a human.

Another aspect of the invention provides a method of treating a mammal suffering from cancer, diabetic retinopathy, age-related macular degeneration, inflammation, stroke, ischemic myocardium, atherosclerosis, macular edema or psoriasis, comprising administering to said patient in need thereof a therapeutically effective amount of a compound of formula (I) or a pharmaceutically acceptable salt thereof in combination with a second therapeutic agent that inhibits VEGF, VEGFR tyrosine kinase or integrin. In one embodiment of this aspect, said second therapeutic agent is a therapeutic antibody. In yet one embodiment of this aspect, said second therapeutic agent is selected from an alkylating agent; a folic acid antagonist; an antimetabolite of nucleic acid metabolism; a pyrimidine analog; 5-fluorouracil; and a purine nucleoside. In another embodiment of this aspect, said mammal is a human. In another embodiment of this aspect, said second therapeutic agent is administered in combination or sequentially with the first therapeutic agent.

Another aspect of the invention provides a method of treating a patient suffering from cancer, diabetic retinopathy, age-related macular degeneration, inflammation, stroke, ischemic myocardium, atherosclerosis, macular edema or psoriasis, comprising administering to said patient in need thereof a therapeutically effective amount of a compound of formula (I) or a pharmaceutically acceptable salt thereof in combination with radiological treatment, including irridation and/or administration of a radioactive substance.

Another aspect of the invention provides a method of treating a patient suffering from cancer, diabetic retinopathy, age-related macular degeneration, inflammation, stroke, ischemic myocardium, atherosclerosis, macular edema or psoriasis, comprising administering to said patient in need thereof a therapeutically effective amount of a compound of formula (I) or a pharmaceutically acceptable salt thereof in combination with at least two of the treatments mentioned above. Such a method can involve the combination a therapeutically effective amount of a compound of formula (I) or a pharmaceutically acceptable salt thereof in combination with any antiangiogenic agent, radiological treatment or chemotherapy.

Further aspects and embodiments of the invention are as defined in the claims.

### BRIEF DESCRIPTION OF THE DRAWINGS

FIG. 1 is a plot of the tumor volume (mL) in mice having received subcutaneously implanted T241 wt mouse fibrosarcoma tumor cells, as a function of days of therapy by oral administration at 25 mg/kg/day of the compound of Example 1 of the invention. This is compared to administration of vehicle only.

### DETAILED DESCRIPTION OF THE INVENTION

The present invention relates to substituted quinoline derivatives, which can be utilized to treat diseases and conditions such as cancer, diabetic retinopathy, age-related macular degeneration, inflammation, stroke, ischemic myocardium, atherosclerosis, macular edema, psoriasis, and the like in mammals.

The preparation of the compounds of the invention lies well within the capability of the person skilled in the art. As an example, a quinoline-3-carboxylic acid ester of the invention may be formed in a six-step procedure wherein, first, a suitable halo aniline derivative is reacted with a suitable mono- or diethylester, the formed intermediate is cyclized to give a 4-halo-quinoline-3-carboxylic acid ester, which is then coupled with a suitable amine, H(R^{c})N—(CH₂)n-R³, to form a substituted secondary or tertiary 4-amino quinoline-3-carboxylic acid ester. The halogen can then be carbonylated, to yield the corresponding amide, —C(O)—NR¹R². In this context, it should be obvious for the one skilled in the art that a substituted sulphonamide, —S(O)₂—NR¹R², can be prepared via reaction of the halogen with sulfite ion, followed by further manipulation to yield the corresponding sulphonamide or corresponding sulfoxide. The quinoline-3-carboxylic acid ester can then be hydrolysed to give the corresponding carboxylic acid, and finally coupled to the appropriate group, —CHR⁵—(CHR⁶)ₘ—R⁴ to give the compound of formula (I). The entire synthesis is illustrated by Reaction Scheme 1. With regard to the below reaction sequence, it should be well within the capability of the person skilled in the art to select suitable reaction components as well as reaction conditions.

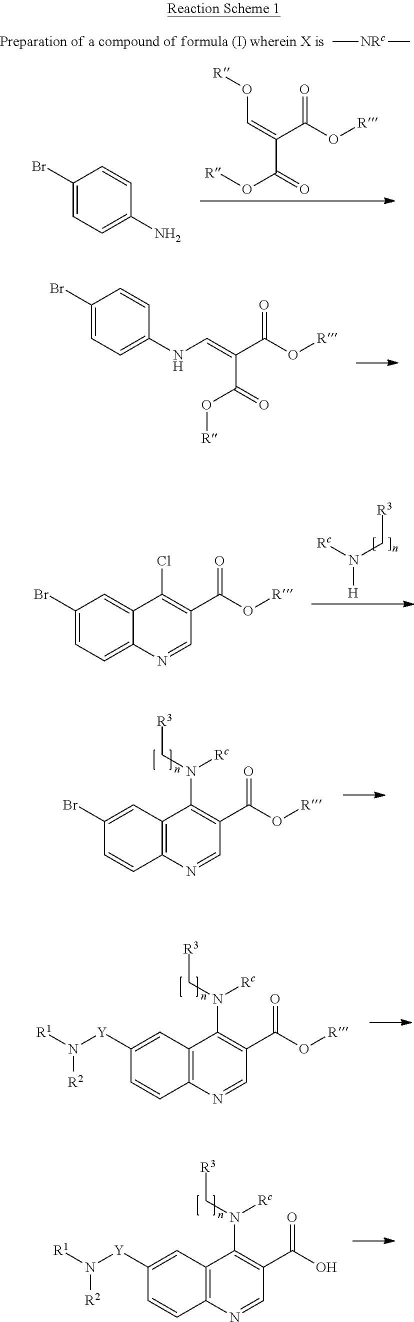

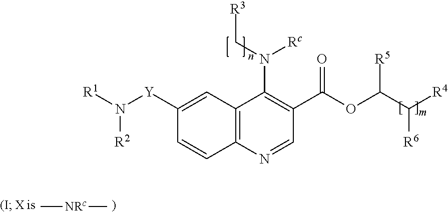

Another synthetic method useful for preparing the inventive compounds is illustrated in Reaction Scheme 2. In this case the synthesis is started from a suitable 6-aniline derivative, —Y— NR¹R², and the amine group, —(R^{c})N(CH₂)ₙR³, is introduced in a later step. The entire synthesis is illustrated by Reaction Scheme 2.

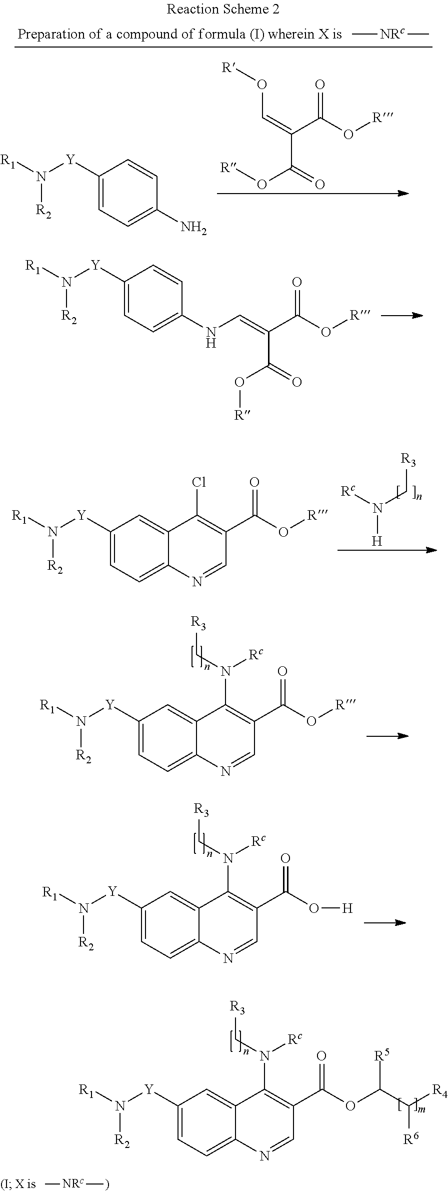

Numerous methods exist in the literature for the synthesis of ethers and sulfides from aryl halides, which should be contemplated when X is O (oxygen) or S. A summary of this work can be found in, for example, Jerry March in Advanced Organic Chemistry, 4^{th} Ed, John Wiley & Sons Inc, New York, 1992, p654-656. An example of a modern synthetic procedure that directly leads to compounds as biaryl ethers can be found in: Evans, D. A.; et al., Tetrahedron Lett. 1998, 39, 2937-2940.

In summary, there are several ways to introduce the groups R¹, R², R³, R⁴, R⁵, R⁶, Y and X, as defined in formula (I), all well known for the one skilled in the art, in order to arrive at the compounds of the invention, and the synthetic routes mentioned herein are not limiting for the invention.

The term “alkyl” as employed herein, alone or as part of another group, refers to an acyclic straight or branched chain radical, unless otherwise specified containing 1, 2, 3, 4, 5, 6, 7 or 8 carbons in the normal chain, which includes methyl, ethyl, n-propyl, n-butyl, n-pentyl, n-hexyl, n-heptyl and n-octyl. Examples of branched chain radicals, not excluding any of the possible isomers not mentioned, are iso-propyl, sec-butyl, iso-pentyl, 3-methylpentyl, 2,3-dimethylhexyl, 3-ethylhexyl, and the like. Unless otherwise specified, the term alkyl also includes a straight or branched alkyl group that contains or is interrupted by a carbocyclyl, exemplified by cyclopropane, as exemplified below:

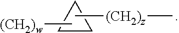

In case the alkyl is interrupted or terminated by a carbocyclyl, the alkyl portions can be attached at any variable point of attachment to the carbocyclyl, including the same ring carbon, as exemplified below:

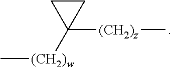

When the alkyl chain is interrupted or terminated by a carbocyclyl, the total number of carbon atoms of the alkyl chain and the carbocyclyl is at most 8. In other words, in the above given example, the sum of z and w is at most 5.

When substituted alkyl is present, this refers to a straight or branched alkyl group as defined above, substituted with 1, 2 or 3 groups of R^{a}. The alkyl group preferably contains 1, 2, 3 or 4 carbons in the normal chain that also can be substituted with 1, 2 or 3 groups of R^{a}, which groups may be the same or different at any available point, as defined with respect to each variable. When such a substituted alkyl group is present, the preferred substitution is halogen such as in —CH₂Cl, —CF₃, —CH₂I, —CHF₂, —CH₂Br, —CH₂F, —CHFCH₂F, —CHFCH₂Cl, —CHFCHClCH₃, —CHClCHBrCH₂CF₃, —CHClCBrICH₂CF₃, —CH₂CH₂CH₂CH₂I, and the like.

The term “alkenyl” as used herein, alone or as part of another group, refers to a straight or branched chain radical, unless otherwise specified containing 2, 3, 4, 5, 6, 7 or 8 carbons, which contains at least one carbon to carbon double bond. Preferably only one carbon to carbon double bond is present, such as in the normal chain vinyl, 2-propenyl, 3-butenyl, 2-butenyl, 4-pentenyl, 3-pentenyl, 2-hexenyl, 3-hexenyl, 2-heptenyl, 3-heptenyl, 4-heptenyl, 3-octenyl, and the like. The alkenyl group preferably contains 2, 3 or 4 carbons in the normal chain. As described above with respect to the “alkyl”, the straight or branched portion of the alkenyl group may be optionally substituted when a substituted alkenyl group is provided. Furthermore, unless otherwise specified, the chain may be interrupted or terminated by a carbocyclyl group, in which case the total number of carbon atoms of the chain and the carbocyclyl is at most 8.

The term “alkynyl” as used herein by itself or as part of another group refers to a straight or branched chain radical, unless otherwise specified containing 2, 3, 4, 5, 6, 7 or 8 carbons, which contains at least one carbon to carbon triple bond. Preferably, only one carbon to carbon triple bond is present, such as in the normal chain 2-propynyl, 3-butynyl, 2-butynyl, 4-pentynyl, 3-pentynyl, 2-hexynyl, 3-hexynyl, 2-heptynyl, 3-heptynyl, 4-heptynyl, 3-octynyl, and the like. The alkynyl group preferably contains 1, 2, 3 or 4 carbons in the normal chain. As described above with respect to the “alkyl”, the straight or branched portion of the alkynyl group may be optionally substituted when a substituted alkynyl group is provided. Furthermore, unless otherwise specified, the chain may be interrupted or terminated by a carbocyclyl group, in which case the total number of carbon atoms of the chain and the carbocyclyl is at most 8.

The term “carbocyclyl” as employed herein alone or as part of another group includes saturated cyclic hydrocarbyl groups or unsaturated (at least 1 double bond) cyclic hydrocarbyl groups, containing at least one ring of in total of 3, 4, 5, 6, 7 or 8 ring carbons, which includes cyclopropyl, cyclobutyl, cyclopentyl, cyclohexyl, cycloheptyl, cyclopentenyl, cyclohexenyl, and the like. The cyclic hydrocarbyl may be monocyclic or bicyclic (i.e. containing two rings of 3 to 8 ring carbons each). As described above with respect to the “alkyl”, the carbocyclyl group may be optionally substituted by 1, 2 or 3 halogens, which may be the same or different.

As used herein, and unless otherwise specified, the term “heterocyclyl” mean a non-aromatic cyclic group that optionally might be unsaturated, containing one or more heteroatom(s) preferably selected from N, O and S, such as a 4 to 10-membered ring system containing at least one heteroatom, e.g. 1-4 heteroatoms. A heterocyclyl e.g. may be, but is not limited to, aziridinyl, azetidinyl, dihydropyranyl, dihydropyridyl, dihydropyrrolyl, dioxolanyl, dioxanyl, dithianyl, dithiolanyl, imidazolidinyl, imidazolinyl, morpholinyl, oxetanyl, oxiranyl, pyrrolidinyl, pyrrolidinonyl, piperidyl, piperazinyl, piperidinyl, pyrazolidinyl, quinuclidinyl, sulfalonyl, 3-sulfolenyl, tetrahydrofuranyl, tetrahydropyranyl, tetrahydropyridyl, thietanyl, thiiranyl, thiolanyl, thiomorpholinyl, trithianyl, tropanyl, 1H-indazolyl and monosaccharide.

The term “halogen” refers to fluorine, chlorine, bromine and iodine, where the preferred halogen radicals are fluorine and chlorine.

As used herein, the term “aryl” means an aromatic group, monocyclic or bicyclic, such as phenyl or naphthyl, and the like. The aryl group is preferably a monocyclic C₆ aryl (i.e. phenyl).

As used herein, the term “heteroaryl” means a mono- or bicyclic heteroaromatic group containing one or more heteroatom(s) preferably selected from N, O and S, such as a 5 to 10-membered ring system containing at least one heteroatom, e.g. 1-4 heteroatoms. Examples of heteroaryl groups are, but are not limited to, pyridyl, quinolinyl, furanyl, thienyl, oxadiazolyl, thiadiazolyl, thiazolyl, oxazolyl, pyrazolyl, triazolyl, tetrazolyl, isoxazolyl, isothiazolyl, isoquinolinyl, naphthyridinyl, imidazolyl, phenazinyl, phenothiazinyl, phthalazinyl, indolyl, pyridazinyl, quinazolinyl, quinolizinyl, quinoxalinyl, tetrahydroisoquinolinyl, pyrazinyl, indazolyl, indolinyl, pyrimidinyl, thiophenetyl, pyranyl, carbazolyl, chromanyl, cinnolinyl, acridinyl, benzimidazolyl, benzodioxanyl, benzodioxepinyl, benzodioxolyl, benzofuranyl, benzothiazolyl, benzobenzoxadiazolyl, benzoxazinyl, benzoxazolyl, benzomorpholinyl, benzoselenadiazolyl, benzothienyl, purinyl, and pteridinyl.

The terms alkyloxy, alkenyloxy and alkynyloxy refer to a radical of the type RO—, wherein R is an alkyl, alkenyl or alkynyl moiety.

The terms alkylthio, alkenylthio, and alkynylthio refer to a radical of the type RS—, wherein R is an alkyl, alkenyl or alkynyl moiety.

The terms alkyl, alkenyl and alkynyl secondary amino refer to a radical of the type RHN—, wherein R is an alkyl, alkenyl or alkynyl moiety.

The terms alkyl, alkenyl and alkynyl tertiary amino refer to a radical of the type RR′N—, wherein R and R′ are each an independently selected alkyl, alkenyl or alkynyl moiety.

The terms alkyl, alkenyl and alkynyl secondary amido refer to radical of the type RHNC(O)—, wherein R is an alkyl, alkenyl or alkynyl moiety.

The terms alkyl, alkenyl and alkynyl tertiary amido refer to a radical of the type RR′NC(O)—, wherein R and R′ are each an independently selected alkyl, alkenyl or alkynyl moiety.

The terms alkyl, alkenyl and alkynyl carbonyl refer to a radical of the type RC(O)—, wherein R is an alkyl, alkenyl or alkynyl moiety.

The terms alkyl, alkenyl and alkynyl sulfonyl refer to a radical of the type RS(O)₂—, wherein R is an alkyl, alkenyl or alkynyl moiety.

The terms alkyl, alkenyl and alkynyl sulfonyloxy refer to a radical of the type RS(O)₂O—, wherein R is an alkyl, alkenyl or alkynyl moiety.

The terms alkyl, alkenyl and alkynyl secondary sulphonamido refer to a radical of the type RHNS(O)₂—, wherein R is an alkyl, alkenyl or alkynyl moiety.

The terms alkyl, alkenyl and alkynyl tertiary sulphonamido refer to a radical of the type RR′NS(O)₂—, wherein R and R′ are each an independently selected alkyl, alkenyl or alkynyl moiety.

The terms alkyl, alkenyl and alkynyl silyl refer to a radical of the type RR′R″Si—, wherein at least one of R, R′, and R″ is an alkyl, alkenyl or alkynyl moiety.

The terms alkyloxy, alkenyloxy, and alkynyloxy carbonyl refer to a radical of the type ROC(O)—, wherein R is an alkyl, alkenyl or alkynyl moiety.

The term oxo group refers to a group consisting of a carbon atom double bonded to an oxygen atom. Thus, a ring system containing an oxo group in the ring, contains a ring carbon atom double bonded to an oxygen atom, i.e. a moiety of formula >C═O.

By the term “unsaturated”, when referring to a bicyclic system, is meant a ring system comprising at least one double or triple bond in at least one ring. Thus, it is contemplated that both rings may be unsaturated or only one ring may be unsaturated, and the other one being saturated. Furthermore, the term “unsaturated bicyclic” also is intended to refer to a non-aromatic bicyclic system comprising a ring that is either unsaturated or saturated fused to a ring that by itself would be aromatic, such as in indane or 4,5-dihydro-1-indole.

Thus, in one embodiment, the invention relates to a compound of formula (I)

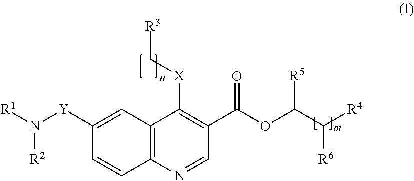

as defined herein above.

In one embodiment, in a compound of formula (I)

n is 0 (zero) or 1;

m is 0 (zero), 1 or 2;

R¹ and R² are independently selected from hydrogen; branched or unbranched C₁-C₈ alkyl, C₂-C₈ alkenyl or C₂-C₈ alkynyl; monocyclic or bicyclic, saturated or unsaturated C₃-C₈ carbocyclyl; and monocyclic or bicyclic, saturated or unsaturated C₁-C₇ heterocyclyl wherein each heteroatom is independently selected from N, O and S; said alkyl, alkenyl, alkynyl, carbocyclyl or heterocyclyl optionally being substituted with 1, 2 or 3 groups R^{a};

R³ is selected from monocyclic or bicyclic C₆-C₁₀ aryl; and monocyclic or bicyclic C₁-C₉ heteroaryl or heterocyclyl, wherein in said heteroaryl and heterocyclyl each heteroatom is independently selected from N, O and S; said aryl, heteroaryl or heterocyclyl optionally being substituted with 1, 2, 3, 4 or 5 groups R^{b};

R⁴ is selected from —NR⁷R⁸; —C(O)NR⁷R⁸; monocyclic or bicyclic C₁-C₉ heteroaryl; and monocyclic or bicyclic, saturated or unsaturated C₁-C₉ heterocyclyl, wherein in said heteroaryl and heterocyclyl each heteroatom independently is selected from N, O and S; said heteroaryl and heterocyclyl optionally being substituted with 1, 2 or 3 groups R^{a};

R⁵ and R⁶ are independently selected from hydrogen; and branched or unbranched C₁-C₄ alkyl, C₂-C₄ alkenyl or C₂-C₄ alkynyl; said alkyl, alkenyl and alkynyl optionally being substituted with 1, 2, or 3 groups independently selected from fluorine and chlorine;

R⁷ is selected from hydrogen; and branched or unbranched C₁-C₄ alkyl, C₂-C₄ alkenyl or C₂-C₄ alkynyl; said alkyl, alkenyl, alkynyl and phenyl optionally being substituted with 1, 2, or 3 groups independently selected from fluorine and chlorine;

R⁸ is selected from hydrogen; branched or unbranched C₁-C₄ alkyl, C₂-C₄ alkenyl or C₂-C₄ alkynyl; monocyclic or bicyclic C₆-C₁₀ aryl; —C(O)OR⁹; and —C(O)R¹⁰; said alkyl, alkenyl, alkynyl or aryl optionally being substituted with 1, 2, or 3 halogen(s);

R⁹ is selected from hydrogen and branched or unbranched C₁-C₄ alkyl, C₂-C₄ alkenyl or C₂-C₄ alkynyl; said alkyl, alkenyl and alkynyl optionally being substituted with 1, 2, or 3 groups independently selected from fluorine and chlorine;

R¹⁰ is selected from hydrogen; branched or unbranched C₁-C₄ alkyl, C₂-C₄ alkenyl or C₂-C₄ alkynyl; and C₆ aryl; said aryl optionally being substituted with 1, 2 or 3 groups R^{a}; and said alkyl, alkenyl and alkynyl optionally being substituted with 1, 2, or 3 groups independently selected from fluorine and chlorine;

Y is selected from —C(O)—; —S(O)—; and —S(O)₂—;

X is selected from —NR^{c}—; —O—; and —S—;

each R^{a} is independently selected from halogen; hydroxy; carbonyl; methoxy; halomethoxy; dihalomethoxy; and trihalomethoxy;

each R^{b} is independently selected from halogen; carboxy; hydroxy; cyano; C₁-C₄ alkyl; C₂-C₄ alkenyl; C₂-C₄ alkynyl; C₁-C₄ alkyloxy; C₂-C₄ alkenyloxy; C₂-C₄ alkynyloxy; C₁-C₄ alkylthio; C₂-C₄ alkenylthio; C₂-C₄ alkynylthio; C₁-C₄ alkyl; C₂-C₄ alkenyl or C₂-C₄ alkynyl secondary or tertiary amino; C₁-C₄ alkyl, C₂-C₄ alkenyl or C₂-C₄ alkynyl secondary or tertiary amido; C₁-C₄ alkyl, C₂-C₄ alkenyl or C₂-C₄ alkynyl carbonyl; C₁-C₄ alkyl, C₂-C₄ alkenyl or C₂-C₄ alkynyl sulfonyl; C₁-C₄ alkyl, C₂-C₄ alkenyl or C₂-C₄ alkynyl sulfonyloxy; C₁-C₄ alkyl, C₂-C₄ alkenyl or C₂-C₄ alkynyl secondary or tertiary suiphonamido; C₁-C₄ alkyl, C₂-C₄ alkenyl or C₂-C₄ alkynyl silyl; and C₁-C₄ alkyloxy, C₂-C₄ alkenyloxy, or C₂-C₄ alkynyloxy carbonyl;

wherein any alkyl, alkenyl and alkynyl moiety optionally is substituted with 1, 2 or 3 groups independently selected from halogen, hydroxy, methoxy, halomethoxy, dihalomethoxy and trihalomethoxy; and

R^{c} is selected from hydrogen; and branched or unbranched C₁-C₄ alkyl, C₂-C₄ alkenyl or C₂-C₄ alkynyl;

or a pharmaceutically acceptable salt thereof.

In one embodiment, in a compound according to formula (I), any alkyl, alkenyl, or alkynyl group having a number of p (p being an integer of 4 to 8) carbon atoms, optionally and independently from any other alkyl, alkenyl or alkynyl group present in the compound, includes a carbocyclic portion of a number of q (q being an integer of 3 to 7 and q being less than p) carbon atoms, which carbocyclic portion may be located so as to interrupt or terminate the straight or branched chain of the alkyl, alkenyl, or alkynyl group, whereby the number of carbon atoms in the straight or branched chain of the alkyl, alkenyl or alkynyl group equals p-q.

In another embodiment, in a compound according to formula (I), any alkyl, alkenyl, or alkynyl group having p carbon atoms has all p carbon atoms in the straight or branched chain portion, i.e. does not include any terminating or interrupting carbocyclic portion.

In a compound of formula (I), the number n of carbon atoms linking the moieties R³ and X is 0 or 1. In one embodiment, n is 0, in which case the compound of formula (I) may be represented by formula (Ia):

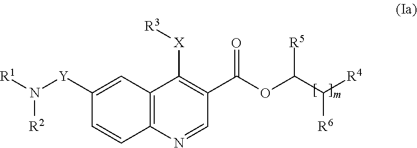

In formula (I), R¹ and R² are independently selected from hydrogen; branched or unbranched C₁-C₈ alkyl, C₂-C₈ alkenyl or C₂-C₈ alkynyl; monocyclic or bicyclic, saturated or unsaturated C₃-C₈ carbocyclyl; and monocyclic or bicyclic, saturated or unsaturated C₁-C₇ heterocyclyl wherein each heteroatom is independently selected from N, O and S; said alkyl, alkenyl, alkynyl, carbocyclyl or heterocyclyl optionally being substituted with 1, 2 or 3 groups R^{a}, e.g 1 or 2 groups R^{a}, or 1 group R^{a}, or being unsubstituted.

In one embodiment of the invention, R¹ and R² are independently selected from hydrogen and branched or unbranched C₁-C₈ alkyl, C₂-C₈ alkenyl or C₂-C₈ alkynyl, e.g. hydrogen and C₁-C₄ alkyl, C₂-C₄ alkenyl or C₂-C₄ alkynyl; said alkyl, alkenyl and alkynyl optionally being substituted with 1, 2 or 3 groups R^{a}. In particular, R¹ and R² may be independently selected from hydrogen and branched or unbranched C₁-C₈ alkyl, e.g. hydrogen and C₁-C₄ alkyl, said alkyl optionally being substituted with 1, 2 or 3 groups R^{a} selected from halogen.

In another embodiment R¹ and R² are independently selected from hydrogen, C₁-C₄ alkyl, C₂-C₄ alkenyl and C₂-C₄ alkynyl, e.g. R¹ and R² are independently selected from hydrogen and C₁-C₄ alkyl, such as hydrogen and C₁-C₃ alkyl, e.g. hydrogen and methyl.

In one embodiment R¹ is hydrogen and R² is as defined herein above, but is not hydrogen; for example, R¹ is hydrogen and R² is C₁-C₃ alkyl, e.g methyl.

In formula (I), R³ is selected from monocyclic or bicyclic C₆-C₁₀ aryl; and monocyclic or bicyclic C₁-C₉ heteroaryl or heterocyclyl, wherein in said heteroaryl and heterocyclyl each heteroatom is independently selected from N, O and S; said aryl, heteroaryl and heterocyclyl optionally being substituted with 1, 2, 3, 4 or 5 groups R^{b}.

In one embodiment R³ is selected from monocyclic C₆ aryl; monocyclic C₁-C₅ heteroaryl and monocyclic C₁-C₅ heterocyclyl, wherein in said heteroaryl and heterocyclyl each heteroatom is independently selected from N, O and S; said aryl, heteroaryl and heterocyclyl optionally being substituted with 1, 2, 3, 4 or 5 groups R^{b}.

In still another embodiment R³ is selected from monocyclic C₆ aryl; and monocyclic C₁-C₅ heteroaryl, wherein in said heteroaryl each heteroatom is independently selected from N, O and S; said aryl and heteroaryl optionally being substituted with 1, 2, 3, 4 or 5 groups R^{b}.

In one embodiment R³ is selected from monocyclic or bicyclic C₆-C₁₀ aryl, said aryl optionally being substituted with 1, 2, 3, 4 or 5 groups R^{b}.

In another embodiment R³ is a monocyclic C₆ aryl (phenyl), optionally being substituted with 1, 2, 3, 4 or 5 groups R^{b}. Thus, in this embodiment, the compound of formula (I) may be represented by formula (Ib):

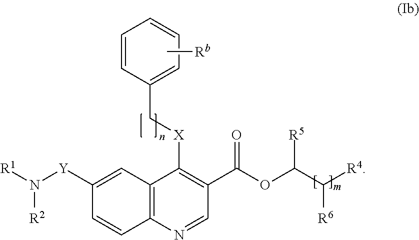

Furthermore, in the embodiment where R³ is a monocyclic C₆ aryl (phenyl), optionally being substituted with 1, 2, 3, 4 or 5 groups R^{b}, a compound of formula (Ia) may be represented by formula (Ic):

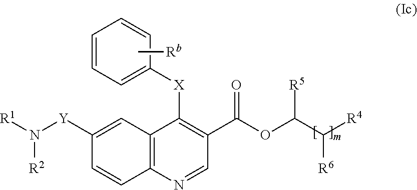

In one embodiment, where R³ is phenyl, it is substituted with a group R^{b} in para position, relative to the bond or chain connecting R³ to X. In one particular embodiment, R³ is a phenyl substituted with 1 R^{b}, in para position relative to the bond or chain connecting R³ to X.

In any of the above embodiments, the number of groups R^{b} e.g. is 1-4, or 1-3, such as 1-2, in particular 1.

In a compound of formula (I), R⁴ is selected from —OC(O)R⁷; —C(O)OR⁷; —NR⁷R⁸; —C(O)NR⁷R⁸; monocyclic or bicyclic C₁-C₉ heteroaryl; and monocyclic or bicyclic, saturated or unsaturated C₁-C₉ heterocyclyl, wherein said heteroaryl and heterocyclyl optionally contains an oxo group in the ring, and wherein in said heteroaryl and heterocyclyl each heteroatom independently is selected from N, O and S; said heteroaryl and heterocyclyl optionally being substituted with 1, 2 or 3 groups R^{a}.

In one embodiment there is provided compounds of formula (I), wherein R⁴ is selected from —NR⁷R⁸; —C(O)NR⁷R⁸; monocyclic or bicyclic C₁-C₉ heteroaryl; and monocyclic or bicyclic, saturated or unsaturated C₁-C₉ heterocyclyl, and wherein in said heteroaryl and heterocyclyl each heteroatom independently is selected from N, O and S; said heteroaryl and heterocyclyl optionally being substituted with 1, 2 or 3 groups R^{a}.

In this embodiment any monocyclic moiety of R⁴ may be e.g. 5- or 6-membered, while any bicyclic moiety of R⁴ may be e.g. 9- or 10-membered; and any monocyclic or bicyclic moiety may contain e.g. 1-4 heteroatoms, such as 1-3 heteroatoms, e.g. 1 or 2 heteroatoms, which heteroatoms e.g. are selected from N and O.

In one embodiment, R⁴ is selected from —NR⁷R⁸; —C(O)NR⁷R⁸; monocyclic C₁-C₄ heteroaryl, and monocyclic, saturated or unsaturated C₁-C₄ heterocyclyl, as defined herein above.

In one embodiment, R⁴ is selected from —NR⁷R⁸; —C(O)NR⁷R⁸; monocyclic 5-6 membered C₁-C₄ heteroaryl, and monocyclic, saturated or unsaturated 5-6 membered C₁-C₄ heterocyclyl, comprising 1-4, 1-3, or 2 heteroatoms independently selected from N, O and S, e.g. N and O.

In the embodiment where R⁴ is —NR⁷R⁸ the compound of formula (I) may be represented by formula (Id):

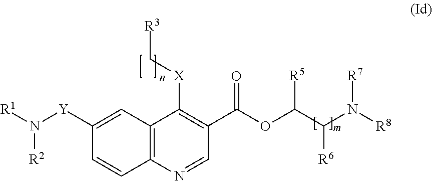

In the embodiment where R⁴ is —C(O)NR⁷R⁸ the compound of formula (I) may be represented by formula (Ie):

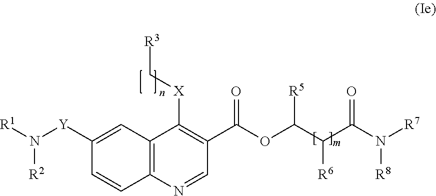

In another embodiment, R⁴ is a monocyclic or bicyclic C₁-C₉ heteroaryl or a monocyclic or bicyclic, saturated or unsaturated C₁-C₉ heterocyclyl, wherein said heteroaryl and heterocyclyl optionally contains an oxo group in the ring, and wherein in said heteroaryl and heterocyclyl each heteroatom independently is selected from N, O and S; and said heteroaryl and heterocyclyl optionally being substituted with 1, 2 or 3 groups R^{a}, e.g. 1 or 2 groups R^{a}, such as 1 group R^{a}. For example, R⁴ may be a 5-10 membered monocyclic or bicyclic C₁-C₉ heteroaryl or a 5-10 membered monocyclic or bicyclic, saturated or unsaturated C₁-C₉ heterocyclyl, said heteroaryl or heterocyclyl containing 1-4 heteroatoms independently selected from N, O and S, e.g. from N and O.

In another embodiment there is provided compounds of formula (I), wherein R⁴ is a monocyclic C₁-C₄ heteroaryl; or a monocyclic saturated or unsaturated C₁-C₄ heterocyclyl, wherein the heteroatoms independently are selected from N, O and S. For example, R⁴ may be a 5-6 membered monocyclic heteroaryl or a 5-6 membered monocyclic saturated or unsaturated heterocyclyl, e.g. containing 1-4 or 1-3, e.g. 1 or 2 heteroatoms independently selected from N, O and S, e.g. N and O, such as imidazolyl, 1,3-dioxolyl or morpholinyl.

In an embodiment where R⁴ is monocyclic C₁-C₄ heteroaryl, or monocyclic, saturated or unsaturated C₁-C₄ heterocyclyl, wherein the heteroatoms independently are selected from N, O and S, the compound of formula (I) may be represented by the formula (If):

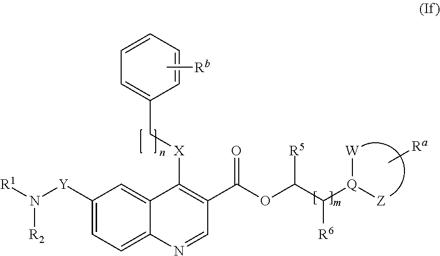

wherein the curbed line:

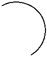

linking Z and W represents a saturated or unsaturated chain of covalently bound atoms independently selected from C (carbon) and heteroatoms, e.g. N, O or S, thus forming a ring structure; Q is selected from C (carbon) and N; W and Z are independently selected from C (carbon), N, O and S.

In one embodiment of a compound of formula (If), the chain of atoms linking W and Z contains 2 to 4 atoms, e.g. 2 to 3 atoms. In a particular embodiment the ring is substituted by one or several radical groups selected from R^{a}. In another embodiment, the ring contains an oxo group.

In a compound of formula (I), R⁵ and R⁶ are independently selected from hydrogen; and branched or unbranched C₁-C₄ alkyl, C₂-C₄ alkenyl or C₂-C₄ alkynyl; said alkyl, alkenyl and alkynyl optionally being substituted with 1, 2, or 3 groups independently selected from fluorine and chlorine.

In one embodiment, R⁵ and R⁶ are independently selected from hydrogen; and branched or unbranched C₁-C₄ alkyl, e.g. C₁-C₃ alkyl, for example methyl, optionally substituted with 1, 2, or 3, e.g. 1 or 2 groups, independently selected from fluorine and chlorine. In one embodiment both R⁵ and R⁶ are hydrogen, in another embodiment only one of R⁵ and R⁶ is hydrogen and the other one is as defined herein above. For example, R⁵ is methyl and R⁶ is hydrogen.

In one embodiment, m is 0. In one particular embodiment, m is 0 and R⁵ is selected from hydrogen; and branched or unbranched C₁-C₄ alkyl, C₂-C₄ alkenyl or C₂-C₄ alkynyl; said alkyl, alkenyl and alkynyl optionally being substituted with 1, 2, or 3 groups independently selected from fluorine and chlorine.

In another embodiment m is 0 and R⁵ is selected from hydrogen; and branched or unbranched C₁-C₄ alkyl, e.g. C₁-C₃ alkyl, for example methyl, optionally substituted with 1, 2, or 3 groups independently selected from fluorine and chlorine.

In one particular embodiment, m is 0 or 1, R⁵ is hydrogen or methyl and R⁶ is hydrogen.

The moiety R⁷ is selected from hydrogen; branched or unbranched C₁-C₄ alkyl, C₂-C₄ alkenyl or C₂-C₄ alkynyl; and phenyl; said alkyl, alkenyl, alkynyl and phenyl optionally being substituted with 1, 2, or 3 groups independently selected from fluorine and chlorine.

In one embodiment, R⁷ is selected from hydrogen; and branched or unbranched C₁-C₄ alkyl, C₂-C₄ alkenyl or C₂-C₄ alkynyl; said alkyl, alkenyl and alkynyl optionally being substituted with 1, 2, or 3 groups independently selected from fluorine and chlorine. For example, R⁷ may be selected from hydrogen and C₁-C₄ alkyl, e.g. methyl.

In another embodiment, R⁷ is selected from hydrogen; branched or unbranched C₁-C₄ alkyl; and phenyl; said alkyl and phenyl optionally being substituted with 1, 2, or 3 groups independently selected from fluorine and chlorine. For example, R⁷ may be selected from hydrogen; C₁-C₄ alkyl, such as methyl; and phenyl.

The moiety R⁸ is selected from hydrogen; branched or unbranched C₁-C₄ alkyl, C₂-C₄ alkenyl or C₂-C₄ alkynyl; monocyclic or bicyclic C₆-C₁₀ aryl; —S(O)₂R⁹; —C(O)OR⁹; and —C(O)R¹⁰; said alkyl, alkenyl, alkynyl and aryl optionally being substituted with 1, 2, or 3 halogen(s).

In one embodiment, R⁸ is selected from hydrogen; branched or unbranched C₁-C₄ alkyl, C₂-C₄ alkenyl or C₂-C₄ alkynyl; monocyclic or bicyclic C₆-C₁₀ aryl; —C(O)OR⁹; and —C(O)R¹⁰; said alkyl, alkenyl, alkynyl or aryl optionally being substituted with 1, 2, or 3 halogen(s).

In one embodiment, R⁸ is selected from branched or unbranched C₁-C₄ alkyl, C₂-C₄ alkenyl or C₂-C₄ alkynyl; —S(O)₂R⁹; —C(O)OR⁹; and —C(O)R¹⁰; said alkyl, alkenyl, alkynyl or aryl optionally being substituted with 1, 2, or 3 halogen(s).

In one particular embodiment, R⁸ is selected from C₁-C₄ alkyl, —S(O)₂R⁹; —C(O)OR⁹; and —C(O)R¹⁰, e.g. C₁-C₄ alkyl, such as methyl; —S(O)₂CH₃; —C(O)OCH₃ and —C(O)phenyl.

The moiety R⁹ is selected from hydrogen and branched or unbranched C₁-C₄ alkyl, C₂-C₄ alkenyl or C₂-C₄ alkynyl; said alkyl, alkenyl and alkynyl optionally being substituted with 1, 2, or 3 groups independently selected from fluorine and chlorine.

In one embodiment, R⁹ is selected from hydrogen and branched or unbranched C₁-C₄ alkyl, optionally substituted with 1, 2, or 3 groups independently selected from fluorine and chlorine. For example, R⁹ may be hydrogen or C₁-C₄ alkyl, such as methyl.

The moiety R¹⁰ is selected from hydrogen and branched or unbranched C₁-C₄ alkyl, C₂-C₄ alkenyl or C₂-C₄ alkynyl; and C₆ aryl; said aryl optionally being substituted with 1, 2 or 3 groups R^{a}, e.g. 1 or 2 groups R^{a}, such as 1 group R^{a}; and said alkyl, alkenyl and alkynyl optionally being substituted with 1, 2, or 3 groups, e.g. 1 or 2 groups, independently selected from fluorine and chlorine. In one embodiment, R¹⁰ is selected from branched or unbranched C₁-C₄ alkyl, C₂-C₄ alkenyl or C₂-C₄ alkynyl; and C₆ aryl. For example, R¹⁰ is phenyl.

In a compound of formula (I), each R^{b} is independently selected from halogen; carboxy; hydroxy; cyano; C₁-C₄ alkyl; C₂-C₄ alkenyl; C₂-C₄ alkynyl; C₁-C₄ alkyloxy; C₂-C₄ alkenyloxy; C₂-C₄ alkynyloxy; C₁-C₄ alkylthio; C₂-C₄ alkenylthio; C₂-C₄ alkynylthio; C₁-C₄ alkyl; C₂-C₄ alkenyl or C₂-C₄ alkynyl secondary or tertiary amino; C₁-C₄ alkyl, C₂-C₄ alkenyl or C₂-C₄ alkynyl secondary or tertiary amido; C₁-C₄ alkyl, C₂-C₄ alkenyl or C₂-C₄ alkynyl carbonyl; C₁-C₄ alkyl, C₂-C₄ alkenyl or C₂-C₄ alkynyl sulfonyl; C₁-C₄ alkyl, C₂-C₄ alkenyl or C₂-C₄ alkynyl sulfonyloxy; C₁-C₄ alkyl, C₂-C₄ alkenyl or C₂-C₄ alkynyl secondary or tertiary sulphonamido; C₁-C₄ alkyl, C₂-C₄ alkenyl or C₂-C₄ alkynyl silyl; and C₁-C₄ alkyloxy, C₂-C₄ alkenyloxy, or C₂-C₄ alkynyloxy carbonyl; wherein any alkyl, alkenyl and alkynyl moiety optionally is substituted with 1, 2 or 3 groups independently selected from halogen, hydroxy, methoxy, halomethoxy, dihalomethoxy and trihalomethoxy.

In one embodiment, R^{b} is independently selected from C₁-C₄ alkyl, C₂-C₄ alkenyl, C₂-C₄ alkynyl, C₁-C₄ alkyloxy, C₂-C₄ alkenyloxy, C₂-C₄ alkynyloxy and halogen.

In a still further particular embodiment R^{b} is selected from C₁-C₄ alkyl, C₂-C₄ alkenyl or C₂-C₄ alkynyl, and the other variables as defined as in any of the embodiments above.

In a still further particular embodiment R^{b} is selected from C₁-C₄ alkyloxy, C₂-C₄ alkenyloxy or C₂-C₄ alkynyloxy, and the other variables are as defined as in any of the embodiments above.

In a still further particular embodiment R^{b} is selected from halogen, and the other variables as defined as in any of the embodiments above.

In another embodiment there is provided compounds of formula (I), wherein R^{b} is selected from C₁-C₄ alkyl, C₂-C₄ alkenyl or C₂-C₄ alkynyl, which optionally are substituted with 1, 2 or 3 independently selected halogen(s).

In another embodiment there is provided compounds of formula (I), wherein R^{b} is selected from C₁-C₄ alkyloxy, C₂-C₄ alkenyloxy and C₂-C₄ alkynyloxy, which optionally are substituted with 1, 2 or 3 independently selected halogen(s).

In another embodiment there is provided compounds of formula (I), wherein R^{b} is halogen.

In formula (I), R^{c} is selected from hydrogen; and branched or unbranched C₁-C₄ alkyl, C₂-C₄ alkenyl or C₂-C₄ alkynyl. In one embodiment, R^{c} is selected from hydrogen and branched or unbranched C₁-C₄ alkyl, e.g C₁-C₃ alkyl, such as methyl. For example, R^{c} is hydrogen or methyl, in particular hydrogen.

In one embodiment there is provided compounds of formula (I), wherein Y is —C(O)—. In this embodiment, the compound of formula (I) may be represented by the formula (Ig):

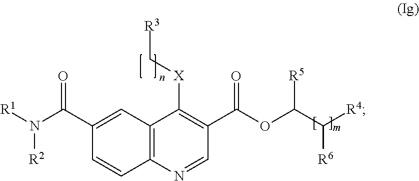

a compound of formula (Ia) may be represented by formula (Ih):

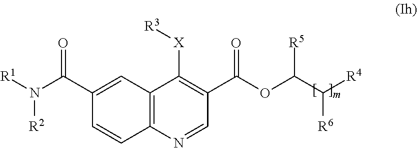

and a compound of formula (Ib) may be represented by formula (Ii):

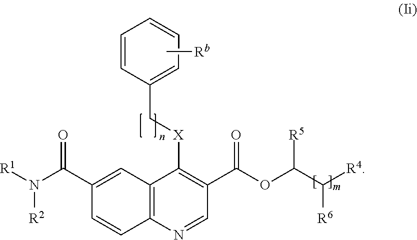

In still another embodiment, Y in formula (I) is C(O) and n is 0 (zero) and R³ is a monocyclic C₆ aryl (phenyl), optionally being substituted with 1, 2, 3, 4 or 5 groups R^{b}. Thus, in this embodiment, the compound of formula (Ih) may be represented by the formula (Ij)

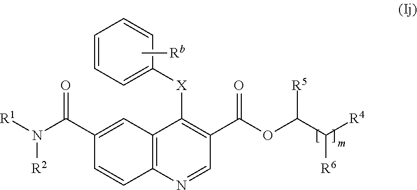

In one embodiment there is provided compounds of formula (I), wherein X represents NR^{c}. In this embodiment, e.g. a compound of formula (Ij) may be represented by formula (Ik):

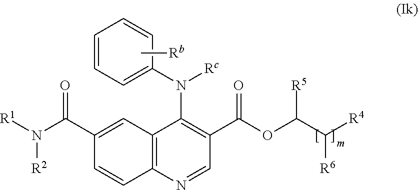

In still another embodiment there is provided compounds of formula (I) wherein R³ is a phenyl substituted with one group R_{b} in para position. In this embodiment, e.g. a compound of formula (Ik) may be represented by the formula (Il):

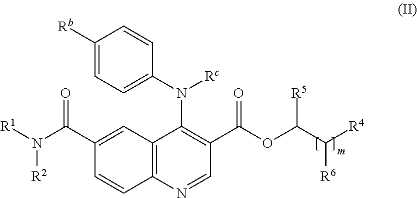

In another embodiment there is provided compounds of formula (I), wherein R¹ and R² are independently selected from hydrogen, C₁-C₄ alkyl, C₂-C₄ alkenyl and C₂-C₄ alkynyl; Y is C(O); X is —NR^{c}—; n is 0 (zero); m is 0 (zero) or 1; R³ is phenyl, optionally being substituted with 1, 2, 3, 4 or 5 groups R^{b}; each R^{b} is independently selected from halogen, C₁-C₄ alkyl, C₂-C₄ alkenyl, C₂-C₄ alkynyl, C₁-C₄ alkyloxy, C₂-C₄ alkenyloxy and C₂-C₄ alkynyloxy, each R^{b}, when different from halogen, independently optionally being substituted with 1, 2 or 3 halogen(s); R⁴ is selected from monocyclic C₁-C₄ heteroaryl and monocyclic, saturated or unsaturated C₁-C₄ heterocyclyl wherein the heteroatoms independently are selected from N, O and S; e.g. a 5- or 6-membered monocyclyl, —OC(O)R⁷; —C(O)OR⁷; —NR⁷R⁸; and —C(O)NR⁷R⁸; each R5 and R6 is hydrogen or methyl; R⁷ represents H, C₁-C₄ alkyl or phenyl; R⁸ is selected from C₁-C₄ alkyl, —S(O)₂R⁹; —C(O)OR⁹ and —C(O)R¹⁰ ; R⁹ represents C₁-C₄ alkyl; R¹⁰ represents C₆ aryl; and pharmaceutically acceptable salts thereof.

In another embodiment there is provided compounds of formula (I), wherein R¹ represents hydrogen; R² represents C₁-C₄ alkyl; Y is C(O); X represents NR^{c}; n is 0 (zero); m is 0 (zero) or 1; R^{c} represents hydrogen; R³ represents a monocyclic C₆ aryl, substituted with 1 R^{b}; R^{b} represents halogen or C₁-C₄ alkyloxy; R⁴ represents a monocyclic C₁-C₄ heteroaryl, such as a 5- or 6-membered heteroaryl; and R5 and R6 is hydrogen or methyl.

In another embodiment there is provided compounds of formula (I), wherein R¹ represents hydrogen; R² represents C₁-C₄ alkyl; Y is C(O); X represents NR^{c}; R^{c} represents hydrogen; R³ represents a monocyclic C₆ aryl, substituted with 1 R^{b}; R^{b} represents halogen or C₁-C₄ alkyloxy; n represents 0 (zero); m represents 0 (zero) or 1; R⁴ represents —OC(O)R⁷; —C(O)OR⁷; —NR⁷R⁸; or —C(O)NR⁷R⁸; R5 and R6 is hydrogen or methyl; R⁷ represents H, C₁-C₄ alkyl or phenyl; R⁸ is selected from C₁-C₄ alkyl, —S(O)₂R⁹; —C(O)OR⁹ and —C(O)R¹⁰; R⁹ represents C₁-C₄ alkyl; and R¹⁰ represents C₆ aryl.

In another embodiment there is provided compounds of formula (I), wherein R¹ represents hydrogen; R² represents C₁-C₄ alkyl; Y is C(O); X represents NR^{c}; R^{c} represents hydrogen; n represents 0 (zero); m represents 0 (zero) or 1; R³ represents a monocyclic C₆ aryl, substituted with R^{b}; R^{b} represents halogen or trifluoromethyl; and R⁴ represents a monocyclic C₁-C₄ heteroaryl.

In another embodiment there is provided compounds of formula (I), wherein R¹ represents hydrogen; R² represents C₁-C₄ alkyl; Y is C(O); X represents NR^{c}; R^{c} represents hydrogen; n represents 0 (zero); m represents 0 (zero) or 1; R³ represents a monocyclic C₆ aryl, substituted with R^{b}; R^{b} represents halogen or trifluoromethyl; R⁴ represents —OC(O)R⁷; —C(O)OR⁷; —NR⁷R⁸; —C(O)NR⁷R⁸; R⁷ represents H, C₁-C₄ alkyl or phenyl; R⁸ is selected from C₁-C₄ alkyl, —S(O)₂R⁹; —C(O)OR⁹ and —C(O)R¹⁰; R⁹ represents C₁-C₄ alkyl; and R¹⁰ represents C₆ aryl.

In another embodiment there is provided compounds of formula (I), wherein R¹ and R² are independently selected from hydrogen, C₁-C₄ alkyl, C₂-C₄ alkenyl and C₂-C₄ alkynyl; Y is C(O); n is 0 (zero); R³ is phenyl, optionally being substituted with 1, 2, 3, 4 or 5 groups R^{b}; each R^{b} is independently selected from C₁-C₄ alkyl, C₂-C₄ alkenyl, C₂-C₄ alkynyl, C₁-C₄ alkyloxy, C₂-C₄ alkenyloxy and C₂-C₄ alkynyloxy, each R^{b} independently optionally being substituted with 1, 2 or 3 halogen(s); R⁴ is selected from monocyclic C₁-C₄ heteroaryl and monocyclic, saturated or unsaturated C₁-C₄ heterocyclyl wherein the heteroatoms independently are selected from N, O and S; —NR⁷R⁸ and —C(O)NR⁷R⁸; X is —NR^{c}—; and each R5 and R6 is hydrogen.

In another embodiment there is provided compounds of formula (I), wherein R¹ represents hydrogen; R² represents C₁-C₄ alkyl; X represents NR^{c}; R^{c} represents hydrogen; R³ represents a monocyclic C₆ aryl, substituted with 1 R^{b}; R^{b} represents C₁-C₄ alkyloxy; n represents 0 (zero); m represents 0 (zero) or 1; and R⁴ represents a monocyclic C₁-C₄ heteroaryl.

In another embodiment there is provided compounds of formula (I), wherein R¹ represents hydrogen; R² represents C₁-C₄ alkyl; X represents NR^{c}; R^{c} represents hydrogen; R³ represents a monocyclic C₆ aryl, substituted with 1 R^{b}; R^{b} represents C₁-C₄ alkyloxy; n represents 0 (zero); m represents 0 (zero) or 1; R⁴ represents —NR⁷R⁸ or —C(O)NR⁷R⁸; R⁷ represents C₁-C₄ alkyl; R⁸ is selected from C₁-C₄ alkyl, —C(O)OR⁹ and —C(O)R¹⁰; R⁹ represents C₁-C₄ alkyl; and R¹⁰ represents C₆ aryl.

In another embodiment there is provided compounds of formula (I), wherein R¹ represents hydrogen; R² represents C₁-C₄ alkyl; X represents NR^{c}; R^{c} represents hydrogen; R³ represents a monocyclic C₆ aryl, substituted with R^{b}; R^{b} represents halogen or trifluoromethyl; n represents 0 (zero); m represents 0 (zero) or 1; and R⁴ represents a monocyclic C₁-C₄ heteroaryl.

In another embodiment there is provided compounds of formula (I), wherein R¹ represents hydrogen; R² represents C₁-C₄ alkyl; X represents NR^{c}; R^{c} represents hydrogen; R³ represents a monocyclic C₆ aryl, substituted with R^{b}; R^{b} represents halogen or trifluoromethyl; n represents 0 (zero); m represents 0 (zero) or 1; R⁴ represents NR⁷R⁸ or —C(O)NR⁷R⁸; R⁷ represents C₁-C₄ alkyl; R⁸ is selected from C₁-C₄ alkyl, —C(O)OR⁹ and —C(O)R¹⁰; R⁹ represents C₁-C₄ alkyl; and R¹⁰ represents C₆ aryl.

In another embodiment there is provided a compound of formula (I), which is:

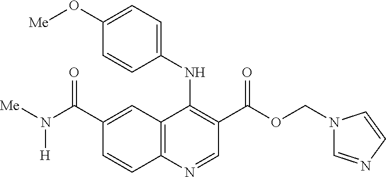

(1H-imidazol-1-yl)methyl 4-(4-methoxyphenylamino)-6-(methylcarbamoyl)quinoline-3 -carboxylate;

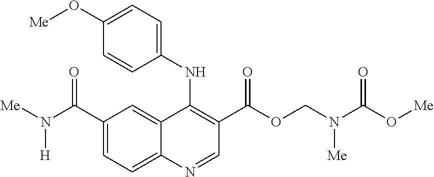

(methoxycarbonyl(methyl)amino)methyl 4-(4-methoxyphenylamino)-6-(methylcarbamoyl(quinoline-3-carboxylate;

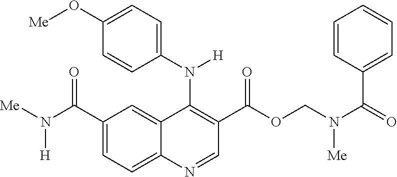

(N-methylbenzamido)methyl 4-(4-methoxyphenylamino)-6-(methylcarbamoyl)quinoline-3-carboxylate;

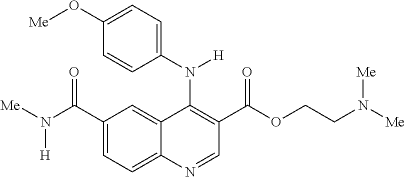

2-(dimethylamino)ethyl 4-(4-methoxyphenylamino)-6-(methylcarbamoyl)quinoline-3-carboxylate;

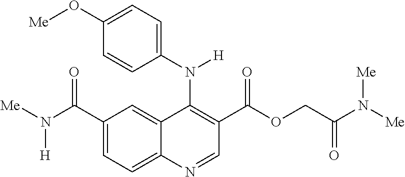

2-(dimethylamino)-2-oxoethyl 4-(4-methoxyphenylamino)-6-(methylcarbamoyl)quinoline-3-carboxylate;

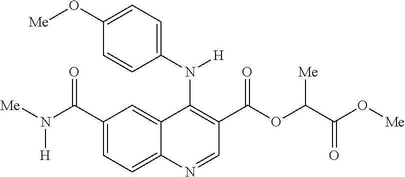

(2-Methoxy-1-methyl-2-oxo-ethyl) 4-[(4-methoxyphenyl)amino]-6-(methylcarbamoyl)quinoline-3-carboxylate;

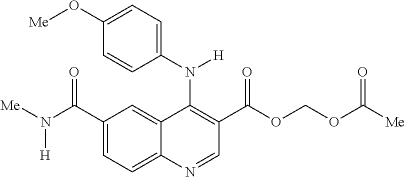

Acetoxymethyl 4-[(4-methoxyphenyl)amino]-6-(methylcarbamoyl)quinoline-3-carboxylate;

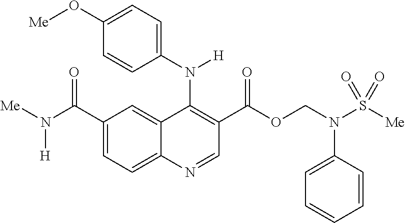

(Methylsulfonyl(phenyl)amino)methyl 4-[(4-methoxyphenyl)amino]-6-(methylcarbamoyl)quinoline-3-carboxylate;

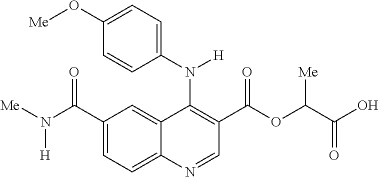

2-[4-[(4Methoxyphenyl)amino]-6-(methylcarbamoyl)quinoline-3-carbonyl]oxypropanoic acid;

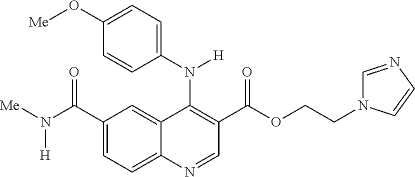

2-Imidazol-1-ylethyl 4-[(4-methoxyphenyl(amino]-6-(methylcarbamoyl)-quinoline-3-carboxylate;

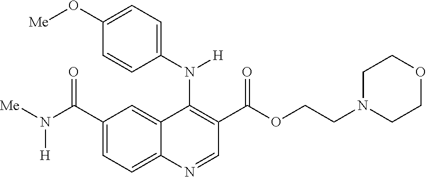

2-Morpholinoethyl 4-[(4-methoxyphenyl(amino]-6-(methylcarbamoyl)-quinoline-3-carboxylate;

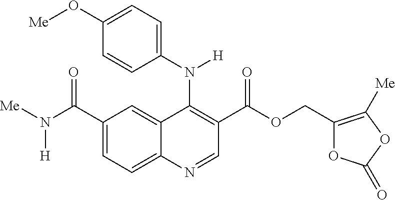

(5-Methyl-2-oxo-1,3-dioxol-4-yl)methyl 4-[(4-methoxyphenyl)amino]-6-(methylcarbamoyl)quinoline-3-carboxylate;

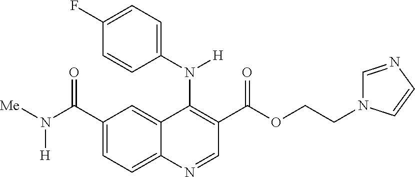

4-(4-Fluoro-phenylamino)-6-methylcarbamoyl-quinoline-3-carboxylic acid 2-imidazol-1-yl-ethylester;

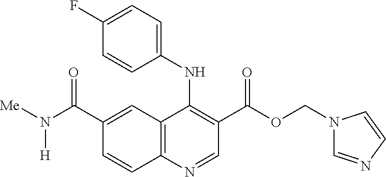

4-(4-Fluoro-phenylamino)-6-methylcarbamoyl-quinoline-3-carboxylic acid imidazol-1-yl-methylester;

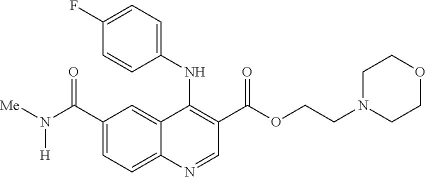

2-Morpholinoethyl 4-[(4-fluorophenyl)amino]-6-(methylcarbamoyl)quinoline-3-carboxylate,

or a pharmaceutically acceptable salt thereof.

It should be understood, that, unless the contrary is indicated or apparent from the context, any reference made herein to a compound of formula (I) also is intended to refer to a compound of formula (Ia), (Ib), (Ic), (Id), (Ie), (If), (Ig), (Ih), (Ii), (Ij), (Ik), or (Il) which are embodiments comprised within the scope of formula (I).

The compounds of the invention can be present as salts, which are also within the scope of this invention. Pharmaceutically acceptable (i.e., non-toxic, physiologically acceptable) salts are preferred.

For example, the inventive compounds can form acid addition salts, e.g. at the amino function. These may be formed, for example, with strong inorganic acids, such as mineral acids, for example sulfuric acid, phosphoric acid or a hydrohalic acid; strong organic carboxylic acids, such as alkanecarboxylic acids of 1 to 4 carbon atoms which are unsubstituted or substituted, for example, by halogen, for example acetic acid, saturated or unsaturated dicarboxylic acids, for example oxalic, malonic, succinic, maleic, fumaric, phthalic or terephthalic acid, hydroxycarboxylic acids, for example ascorbic, glycolic, lactic, malic, tartaric or citric acid, amino acids, (for example aspartic or glutamic acid or lysine or arginine), or benzoic acid, or with organic sulfonic acids, such as (C₁-C₄) alkyl or arylsulfonic acids which are unsubstituted or substituted, for example by halogen, for example methyl- or p-toluene-sulfonic acid. Corresponding acid addition salts can also be formed having, if desired, an additionally present basic center.

The compounds of formula I having at least one acid group (for example C(O)OH) can also form salts with bases. Suitable salts with bases are, for example, metal salts, such as alkali metal or alkaline earth metal salts, for example sodium, potassium or magnesium salts, or salts with ammonia or an organic amine, such as morpholine, thiomorpholine, piperidine, pyrrolidine, mono-, di- or tri-lower alkylamine, for example ethyl, tert-butyl, diethyl, diisopropyl, triethyl, tributyl or dimethyl-propylamine, or a mono, di or trihydroxy lower alkylamine, for example mono-, di- or triethanolamine. Corresponding internal salts may furthermore be formed. Salts that are unsuitable for pharmaceutical uses but which can be employed, for example, for the isolation or purification of free compounds of formula I or their pharmaceutically acceptable salts are also included.

The present invention also includes prodrugs. In fact, the esters of formula I display improved uptake in vivo and are hydrolyzed to their corresponding carboxylic acids in vivo. The term “prodrug” is intended to represent a compound bonded to a carrier, which prodrug is capable of releasing the active ingredient when the prodrug is administered to a mammalian subject. Release of the active ingredient occurs in vivo. Prodrugs of compounds of the invention include compounds wherein a hydroxyl, amino, carboxylic, or a similar group is modified. Examples of prodrugs include, but are not limited to, esters (e.g. acetate, formate, and benzoate derivatives), carbamates (e.g., N,N-dimethylaminocarbonyl of hydroxyl or amino functional groups of the present invention), amides (e.g., trifluoroacetylamino, acetylamino, and the like), and the like.

The compounds of the invention may be administered as is or as an alternative prodrug, for example in the form of an in vivo hydrolysable ester or in vivo hydrolysable amide. An in vivo hydrolysable ester of a compound of the invention containing carboxy or hydroxyl group is, for example, a pharmaceutically acceptable ester which is hydrolysed in the human or animal body to produce the parent acid or alcohol. Suitable pharmaceutically acceptable esters for carboxy include C₁-C₆ alkyloxymethyl esters (e.g., methoxymethyl) C₁-C₆ alkanoyloxymethyl esters (e.g., pivaloyloxymethyl), phthalidyl esters, C₃-C₈ cycloalkyloxycarbonyloxy-C₁-C₆ alkyl esters (e.g. 1-cyclohexylcarbonyloxyethyl), 1,3-dioxolen-2-onylmethyl esters (e.g., 5-methyl-1,3-dioxolen-2-onylmethyl) and C₁-C₆alkyloxycarbonyloxyethyl esters (e.g., 1-methoxycarbonyloxyethyl) and may be formed at any appropriate carboxy group in the compounds of the invention.

An in vivo hydrolysable ester of a compound of the invention containing a hydroxyl group includes inorganic esters such as phosphate esters and acyloxyalkyl ethers and related compounds which as a result of the in vivo hydrolysis of the ester breakdown to give the parent hydroxy group. Examples of acyloxyalkyl ethers include acetoxymethoxy and 2,2-dimethyl-propionyloxy-methoxy. A selection of in vivo hydrolysable ester forming groups for hydroxy include alkanoyl, benzoyl, phenylacetyl and substituted benzoyl and phenylacetyl, alkoxycarbonyl (to give alkyl carbonate esters), dialkylcarbamoyl and N-(N,N-dialkylamino-ethyl)-N-alkylcarbamoyl (to give carbamates), N,N-dialkylaminoacetyl and carboxyacetyl. Examples of substituents on benzoyl include morpholino and piperazino linked from a ring nitrogen atom via a methylene group to the 3- or 4-position of the benzoyl ring. A suitable value for an in vivo hydrolysable amide of a compound of the invention containing a carboxy group is, for example, an N—C₁-C₆ alkyl or N,N-diC₁-C₆ alkyl amide such as N-methyl, N-ethyl, N-propyl, N,N-dimethyl, N-ethyl-N-methyl or N,N-diethyl amide. Upon administration of a compound of the invention, or an alternative prodrug thereof, the prodrug undergoes chemical conversion by metabolic or chemical processes to yield another compound, for example a salt and/or solvate thereof. Solvates of the compounds of the present invention include, for example hydrates.

An administration of a therapeutic agent of the invention includes administration of a therapeutically effective amount of the agent of the invention. The term “therapeutically effective amount” as used herein refers to an amount of a therapeutic agent to treat or prevent a condition treatable by administration of a composition of the invention. That amount is the amount sufficient to exhibit a detectable therapeutic or preventative or ameliorative effect. The effect may include, for example, treatment or prevention of the conditions listed herein. The precise effective amount for a subject will depend upon the subject's size and general condition, the nature and extent of the condition being treated, recommendations of the treating physician, and the therapeutics or combination of therapeutics selected for administration. Thus, it is not useful to exactly specify an exact effective amount in advance. In the case of oral administration the dosage might, however, vary from about 0.01 mg to about 1000 mg per day of a compound of formula (I) or the corresponding amount of a pharmaceutically acceptable salt thereof.

The composition according to the invention may be prepared for any route of administration, e.g. oral, intravenous, cutaneous or subcutaneous, nasal, intramuscular, or intraperitoneal. The precise nature of the carrier or other material will depend on the route of administration. For parenteral administration, a parenterally acceptable aqueous solution is employed, which is pyrogen free and has requisite pH, isotonicity and stability. Those skilled in the art are well able to prepare suitable solutions and numerous methods are described in the literature.

The pharmaceutically acceptable excipients described herein, for example, vehicles, adjuvants, carriers or diluents, are well-known to those who are skilled in the art and are readily available to the public. The pharmaceutically acceptable carrier may be one that is chemically inert to the active compounds and that has no detrimental side effects or toxicity under the conditions of use. Examples of pharmaceutical formulations can be found in Remington: The Science and Practice of Pharmacy. A. R. Gennaro, Editor. Lippincott, Williams and Wilkins, 20th edition (2000).

All stereoisomers of the compounds of the instant invention are contemplated, either in admixture or in pure or substantially pure form. The compounds of the present invention can have asymmetric centers at any of the carbon atoms including any one of the R substituents. Consequently, compounds of formula I can exist in enantiomeric or diasteromeric forms or in mixtures thereof. The processes for preparation can utilize racemates, enantiomers or diasteromers as starting materials. When diastereomeric or enantiomeric products are prepared, they can be separated by conventional methods, which for example is chromatographic or fractional crystallization.

The effectiveness of the compounds of the invention in preventing or treating disease may be improved by administering the compounds in combination with another agent that is effective for those purposes, such as, but not limited to, another antiangiogenic compounds inhibiting VEGF, VEGFR tyrosine kinase, integrin inhibitors, phototherapies, antibodies against VEGF, or one or more conventional therapeutic agents such as, alkylating agents, folic acid antagonists, anti-metabolites of nucleic acid metabolism, pyrimidine analogs, 5-fluorouracil, purine nucleosides. Such other agents may be present in the composition being administered or may be administered separately. Also, the compounds of the invention are suitably administered serially or in combination with radiological treatments, whether involving irradiation or administration of radioactive substances.

The term antiangiogenic as used herein by itself or as a part of another definition refers to a compound with the ability to inhibit angiogenesis, which is the growth of new blood vessels, e.g. into a solid tumor.

The number of mechanisms for antiangiogenic agents is diverse and may include, but not limited to, compounds that inhibit cell proliferation, inhibit cell migration of endothelial cells, activate immune system, downregulate angiogenesis stimulators, stimulate angiogenesis inhibitor formation, inhibit binding of angiogenesis stimulators, inhibit basement membrane degradation, induce apoptosis of endothelial cells, inhibit survival of endothelial cells, inhibit cell adhesion and inhibit survival of endothelial cells.

The number of compounds or monoclonal antibodies that are antiangiogenic may include, but is not limited to, Avastin® (bevacizumab) carboxyamidotriazole (5-Amino-1-(3,5-dichloro-4-(4-chlorobenzoyl)phenyl)methyl)-1H-1,2,3-triazole-4-carboxamide), TNP-470 ((3R,4S,5S,6R)-5-methoxy-4-[(2R,3R)-2-methyl-3-(3-methyl-2-butenyl)-oxiranyl]-1-oxaspiro-[2,5] oct-6-yl(chloroacetyl) carbamate), CM-101 (a bacterial polysaccharide exotoxin produced by group B Streptococcus (GBS), also referred to as GBS toxin), Germanin® (also known as suramin, CAS number 145-63-1), SU5416 (semaxinib, (3Z)-3-[(3,5-dimethyl-1H-pyrrol-2-yl)methylidene]-1,3-dihydro-2H-indol-2-one), TSP (thrombospondins, a group of secreted proteins with antiangiogenic abilities), angiostatic steroids and heparin in combination, matrix metalloproteinase inhibitors, Angiostatin™, Macugen® (pegaptanib sodium injection), Endostatin™, 2-methoxyestradiol, Tecogalan sodium (DS-4152, a bacterial polysaccharide), prolactin (or luteotropic hormone (LTH), a peptide hormone), linomide (LS-2616, [N-methyl-N-phenyl-1,2-dihydro-4-hydroxy-1-methyl-2-oxo-quinoline-3-carboxamide]) and the like.

The term VEGF (vascular endothelial growth factor) as used herein refers to a sub-family of growth factors, which are platelet-derived growth factor family of cystine-knot growth factors. They are important signaling proteins involved in angiogenesis, as well as vasculogenesis (de novo formation of the embryonic circulatory system).

The term VEGFR tyrosine kinase as used herein refers to the tyrosine kinase receptors that the members of the VEGF family bind to.

The term integrin as used herein by itself or as a part of another definition refers to a family of transmembrane glycoproteins consisting of non-covalent heterodimers. The integrins consist of at least three identified families where each family contains a common beta-subunit combined with one or more distinct alpha-subunits. These receptors participate in cell-matrix and cell-cell adhesion in many physiologically important processes, including oncogenic transformation.

The compounds according to formula (I) will be useful for treating various diseases such as cancer, diabetic retinopathy, age-related macular degeneration, inflammation, stroke, ischemic myocardium, atherosclerosis, macular edema and psoriasis. The treatment may be preventive, palliative or curative.

The compounds of the invention provide a method of treating a mammal suffering from a disease or disorder related to VEGFR tyrosine kinase or integrin activity, comprising administering to said mammal in need thereof, a therapeutically effective amount of a compound of formula (I). The said mammal can be a human.

The compounds of the present invention may be used or administered in combination with one or more additional drugs useful in the treatment of hyperproliferative diseases, e.g. antiangiogenic agents, including both compounds and monoclonal antibodies, and a cytostatic agent. The components may be in the same formulation or in separate formulations for administration simultaneously or sequentially. The compounds of the present invention may also be used or administered in combination with other treatment such as irradiation for the treatment of cancer.

Examples of cytotstatic agents for use as indicated herein above are DNA alkylating compounds, topoisomerase I inhibitors, topoisomerase II inhibitors, compounds interfering with RNA and DNA synthesis, compounds polymerising the cytoskeleton, and compounds depolymerising the cytoskeleton.

The invention is illustrated by the following non-limiting Examples.

### EXAMPLES

### Example 1

### (1H-imidazol-1-yl)methyl 4-(4-methoxyphenylamino)-6-(methylcarbamoyl)-quinoline-3-carboxylate

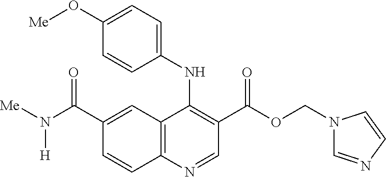

(a) Preparation of intermediary compound diethyl 2-((4-bromophenylamino)methylene)-malonate:

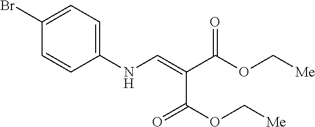

4-Bromoaniline (10 g) and diethoxymethylene malonate (12.6 g) were heated at 150° C. for 3 hours in a sealed tube. The reaction mixture was then cooled and diluted with n-hexane when the solid product precipitated out. This solid was filtered, washed several times with n-hexane and dried under vacuum to afford 17.8 g of 2-[(4-bromo-phenylamino)methylene]malonic acid diethyl ester. ¹H NMR (300 MHz, CDCl₃) δ 11.03 (d, 1H, J=13 Hz, —NH—), 8.48 (d, 1H, J=13 Hz, —CH═C), 7.49 (m, 2H, aromatic), 7.10-7.01 (m, 2H, aromatic), 4.42-4.22 (m, 4H, —CH₂—CH₃), 1.45-1.26 (m, 6H, —CH₂—CH₃); LC-MS (m/z) 343.9 (M+1).

(b) Preparation of intermediary compound 6-bromo-4-chloroquinoline-3-carboxylic acid ethyl ester:

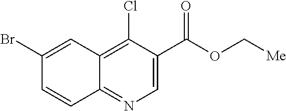

2-[(4-Bromophenylamino)methylene]malonic acid diethyl ester (5 g) was heated with POCl₃ (phosphoryl chloride, 31.5 mL) at 150° C. in a sealed tube for about 6 h. The excess POCl₃ was removed by rotavapor and the crude mixture was diluted with dichloromethane. The dichloromethane extract was washed with aqueous sodium hydroxide solution (10%), dried over sodium sulphate and purified by column chromatography (Silica gel, hexane/ethyl acetate 80:20) to give 2.3 g of 6-bromo-4-chloroquinoline-3-carboxylic acid ethyl ester. ¹H NMR (300 MHz, CDCl₃) δ 9.22 (s, 1H, aromatic), 8.60 (d, 1H, J=2.1 Hz, aromatic), 8.04 (d, 1H, J=9 Hz, aromatic), 7.95-7.85 (m, 1H, aromatic), 4.53 (q, 2H, J=7 Hz, —CH₂—), 1.50 (t, 3H, J=7 Hz, —CH₃); LC-MS (m/z) 315.8 (M+1).

(c) Preparation of intermediary compound ethyl 6-bromo-4-[(4-methoxyphenyl)-amino]quinoline-3-carboxylate:

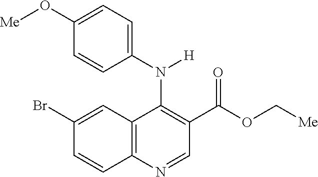

p-Anisidine (0.43 g) and 6-bromo-4-chloroquinoline-3-carboxylic acid ethyl ester (1.0 g) were mixed in dioxane and irradiated in a microwave reactor at 150° C. for 30 minutes. The reaction mixture was diluted with petroleum ether. The solid product obtained was filtered and dried to give 1.3 g of ethyl 6-bromo-4-[(4-methoxyphenyl)amino]quinoline-3-carboxylate. ¹H NMR (300 MHz, CDCl₃) δ 11.41 (s, 1H, —NH—), 9.22 (s, 1H, aromatic), 8.20 (d, 1H, J=8.2 Hz, aromatic), 7.77 (d, 1H, J=8.2 Hz, aromatic), 7.64 (s, 1H, aromatic), 7.15 (d, 2H, J=8.1 Hz, aromatic), 6.99 (d, 2H, J=8.1 Hz, aromatic), 4.47 (q, 2H, J=7 Hz, —CH₂—), 3.89 (s, 3H, —OCH₃), 1.47 (t, 3H, J=7 Hz, —CH₃); LC-MS (m/z) 401.0 (M+1).

(d) Preparation of intermediary compound ethyl 4-[(4-methoxyphenyl)amino]-6-(methyl-carbamoyl)quinoline-3-carboxylate:

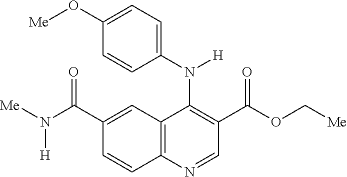

Ethyl 6-bromo-4-[(4-methoxyphenyl)amino]quinoline-3-carboxylate (0.25 g, 0.62 mmol) was added to tetrahydrofuran followed by trans-di(μ-acetato)-bis[o-(di-o-tolylphosphino)-benzyl]dipalladium(II) (Herrmann's palladacycle, 0.031 mmol), [(t-Bu)₃PH]BF₄ (tri tertiarybutyl phosphonium hexafluoborate) (0.125 mmol), molybdenum hexacarbonyl (Mo(CO)₆, 1.246 mmol), methylamine (1.5 equiv., 2 N in THF) and 1,8-diazabicyclo[5.4.0]undec-7-ene (DBU, 1.869 mmol). The reaction mixture was irradiated at 130° C. for 5 minutes in a microwave reactor. The reaction mixture was concentrated and then purified on column (silica gel, dichloromethane/methanol 98:2) to give ethyl 4-[(4-methoxyphenyl)amino]-6-(methylcarbamoyl)-quinoline-3-carboxylate in quantitative yield. ¹H NMR (300 MHz, CDCl₃) δ 10.96 (s, 1H, —NH—) 9.24 (s, 1H, aromatic), 8.14-7.98 (m, 2H, aromatic), 7.73 (s, 1H, aromatic), 7.16 (d, 2H, J=9 Hz, aromatic), 6.98 (d, 2H, J=9 Hz, aromatic), 4.46 (q, 2H, J=7 Hz, —CH₂—), 3.87 (s, 3H, —OCH₃), 1.48 (t, 3H, J=7Hz, —CH₃); LC-MS (m/z) 380.0 (M+1).

(e) Preparation of intermediary compound 4-[(4-methoxyphenyl)amino]-6-(methylcarbamoyl)-quinoline-3-carboxylic acid

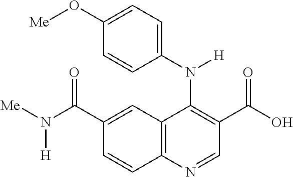

Ethyl 4-[(4-methoxyphenyl)amino]-6-(methylcarbamoyl)quinoline-3-carboxylate (0.2 g, 0.53 mmol) was stirred with lithium hydroxide (85.5 mg) in a mixture of 6 mL of methanol/tetrahydrofuran/water (2:2:2,) overnight. The reaction mixture was concentrated and the aqueous layer was washed with ethyl acetate. The aqueous layers were collected and acidified with aqueous hydrochloric acid and the precipitate formed was filtered and dried to give 0.142 g (77% yield) of 4-[(4-methoxyphenyl)amino]-6-(methylcarbamoyl)quinoline-3-carboxylic acid. ¹H NMR (300 MHz, CD₃OD) δ 9.05 (s, 1H, aromatic), 8.20 (s, 1H, aromatic), 8.12-7.81 (m, 2H, aromatic), 7.27 (d, 2H, J=9.9 Hz, aromatic), 7.06 (d, 2H, J=9.9 Hz, aromatic), 3.88 (s, 1H, —OCH₃), 2.82 (s, 3H, —NCH₃); LC-MS (m/z) 352.0 (M+1).

(f) To a suspension of 4-[(4-methoxyphenyl)amino]-6-(methylcarbamoyl)-quinoline-3-carboxylic acid (1.0 g, 2.8 mmol) in N,N-dimethylformamid (15 mL) at 0° C. was added 1-ethyl-3-(3-dimethyllaminopropyl)carbodiimide hydrochloride (EDC.HCl, 2 g), hydroxybenzotriazole (HOBt, 0.042 g), triethyl amine (4 mL) and 1-hydroxymethyl imidazole (0.34 g, 3.4). The reaction mixture was slowly brought to room temperature and stirred for 5 hours. After aqueous work up, the reaction mixture was extracted, concentrated and dried over anhydrous sodium sulfate to afford the crude product, which was later purified by column chromatography to afford 0.2 g of (1H-imidazol-1-yl)methyl-4-(4-methoxyphenylamino)-6-(methyl-carbamoyl)quinoline-3-carboxylate as a pale yellow solid (17% yield). ¹H NMR (300 MHz, CDCl₃) 10.63 (s, 1H, —CONH—), 9.16 (s, 1H, aromatic), 8.05 (d, 1H, J=8.7 Hz, aromatic), 7.98 (d, 1H, J=8.7 Hz, aromatic), 7.85 (s, 1H, aromatic), 7.77 (s, 1H, aromatic), 7.25 (s,1H, aromatic), 7.19 (d, 1H, J=2.1 Hz, aromatic), 7.00 (s, 1H, aromatic), 6.98 (d, 2H, J=2.1 Hz, aromatic), 6.18 (s, 2H, —CH₂—), 5.49 (bs, 1H, —NH—), 3.88 (s, 3H, —OCH₃), 2.85 (s, 3H, N—CH₃); LC-MS (m/z) 432 (M+1).

### Example 2

### (Methoxycarbonyl(methyl)amino)methyl 4-(4-methoxyphenylamino)-6-(methylcarbamoyl)quinoline-3-carboxylate

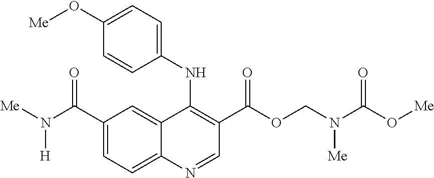

To a suspension of 4-[(4-methoxyphenyl)amino]-6-(methylcarbamoyl)-quinoline-3-carboxylic acid (0.1 g, 0.28 mmol) and N,N-diisopropylethylamine (DIPEA, Hünig's base, 0.12 g) in tetrahydrofuran (15 mL) at 0° C. was added methyl chloromethyl(methyl)carbamate (0.039 g). The reaction mixture was slowly brought to room temperature and stirred overnight. The reaction mixture was then concentrated, extracted with ethyl acetate and purified on column by column (Silica gel, chloroform/methanol, 9:1) to afford 35 mg of (methoxycarbonyl(methyl)-amino)methyl 4-(4-methoxyphenyl-amino)-6-(methylcarbamoyl)-quinoline-3-carboxylate as a solid (28% yield). ¹H NMR (300 MHz, DMSO-d₆) 9.17 (s, 1H, aromatic), 8.45 (s, 1H, aromatic), 8.3 (s, 1H, aromatic), 8.22 (s, 1H, aromatic), 8.19 (s, 1H, aromatic), 8.14 (s,1H, aromatic), 7.25 (d, 1H, J=8 Hz, aromatic), 7.03 (d, 2H, J=9 Hz, aromatic), 6.05 (s, 2H, —CH₂—), 3.80 (s, 3H, —OCH₃), 2.89 (s, 3H, N—CH₃) 2.73 (s, —CONHCH₃), 1.35 (s, 3H, —OCH₃); LC-MS (m/z) 452.9 (M+1).

### Example 3

### (N-methylbenzamido)methyl 4-(4-methoxyphenylamino)-6-(methylcarbamoyl)-quinoline-3-carboxylate

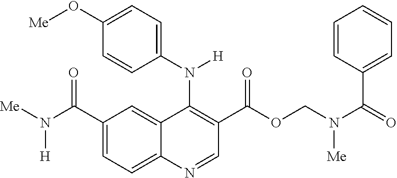

To a suspension of 4-[(4-methoxyphenyl)amino]-6-(methylcarbamoyl)-quinoline-3-carboxylic acid (0.1 g, 0.28 mmol) and N,N-diisopropylethylamine (DIPEA, Hünig's base, 0.12 g) in tetrahydrofuran (15 mL) was stirred at room temperature for 15 minutes. To this solution was added N-(chloromethyl)-N-methylbenzamide (0.051 g) and the reaction mixture was slowly brought to room temperature and stirred for 12 hours. The reaction mixture was then concentrated in vacuo, extracted with ethyl acetate and purified on column (Silica gel, petroleum ether/ethyl acetate) to afford 9 mg of (N-methylbenzamido)methyl 4-(4-methoxyphenyl-amino)-6-(methylcarbamoyl)-quinoline-3-carboxylate as a solid (6% yield). ¹H NMR (300 MHz, CDCl₃) 9.29 (s, 1H, —CONH—), 8.44 (d, 1H, J=4 Hz, aromatic), 8.33 (s, 1H, aromatic), 8.21 (m, 1H, aromatic), 8.05 (s, 1H, aromatic), 7.49 (m, 5H, aromatic), 7.28 (d, 2H, J=8 Hz, aromatic), 7.05 (d, 2H, J=8 Hz, aromatic), 6.27 (s, 2H, —CH₂—), 3.81 (s, 3H, —OCH₃), 2.91 (s, 3H, —NCH₃) 2.5 (s, 3H, -NCH₃); LC-MS (m/z) 498.9 (M+1).

### Example 4

### 2-(dimethylamino)ethyl 4-(4-methoxyphenylamino)-6-(methylcarbamoyl)-quinoline-3-carboxylate

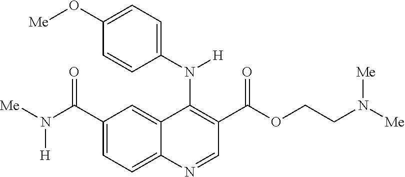

4-[(4-Methoxyphenyl)amino]-6-(methylcarbamoyl)-quinoline-3-carboxylic acid (0.030 g, 0.085 mmol) in N,N-dimethylformamid (4 mL) was mixed in a 10 mL microwave vial. N,N-diisopropylethylamine (DIPEA, Hünig's base, 0.055 g) and 2-chloro-N,N-dimethyl-ethanamine (13.6 mg) were added to the mixture under nitrogen atmosphere. This reaction mixture was irradiated at 150° C. for 15 minutes and the crude reaction mixture was subsequently poured out over crushed ice. The reaction mixture was extracted 3 times with ethyl acetate (50 mL each time), dried over anhydrous sodium sulphate, concentrated in vacuo and recrystallized from n-hexane to afford 10 mg of 2-(dimethylamino)ethyl 4-(4-methoxyphenylamino)-6-(methylcarbamoyl)-quinoline-3-carboxylate as a solid (28% yield). ¹H NMR (300 MHz, methanol-d₄) 9.18 (s, 1H, aromatic), 8.28 (d, 1H, J=1.8 Hz, aromatic), 8.02 (m, 1H, aromatic), 7.91 (m, 1H, aromatic), 7.16 (m, 2H, aromatic), 6.98 (m, 2H, aromatic) 4.52 (t, 2H, J=5.4 Hz, —CH₂—), 3.83 (s, 3H, —OCH₃), 2.84 (m, 5H, N—CH₃ and —CH₂—), 2.38 (s, 6H, —N(CH₃)₂); LC-MS (m/z) 422.9 (M+1).

### Example 5

### 2-(dimethylamino)-2-oxoethyl-4-(4-methoxyphenylamino)-6-(methyl-carbamoyl)quinoline-3-carboxylate

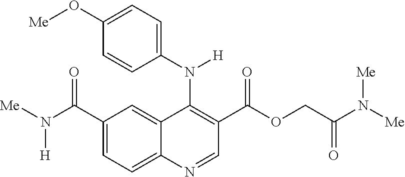

To a suspension of 4-[(4-Methoxyphenyl)amino]-6-(methylcarbamoyl)-quinoline-3-carboxylic acid (0.03 g, 0.085 mmol) and N,N-diisopropylethylamine (DIPEA, Hünig's base, 0.02 g) in tetrahydrofuran (2 mL) at 0° C. was added 2-chloro-N,N-dimethylacetamide (0.015 g) and the reaction mixture was slowly brought to room temperature and stirred overnight. The reaction mixture was concentrated, extracted with ethyl acetate and purified on column (Silica gel, chloroform/methanol 9:1) to afford 8 mg of 2-(dimethylamino)-2-oxoethyl-4-(4-methoxy-phenylamino)-6-(methylcarbamoyl)quinoline-3-carboxylate as a solid (22% yield). ¹H NMR (300 MHz, CDCl₃) 10.75 (s, 1H, —CONH—), 9.14 (s, 1H, aromatic), 8.08 (s, 1H, aromatic), 7.94 (d, 2H, J=7 Hz, aromatic), 7.17 (d, 2H, J=8.7 Hz, aromatic), 6.96 (d, 2H, J=8.7 Hz, aromatic), 6.26 (bs, 1H, —NH—), 4.99 (s, 2H, —CH₂—), 3.86 (s, 3H, —OCH₃), 3.10 (s, —NCH₃), 3.04(s, —NCH₃), 2.89 (s, 3H, —CONHCH₃); LC-MS (m/z) 436.9 (M+1).

### Example 6

### (2-Methoxy-1-methyl-2-oxo-ethyl) 4-[(4-methoxyphenyl)amino]-6-(methylcarbamoyl)quinoline-3-carboxylate

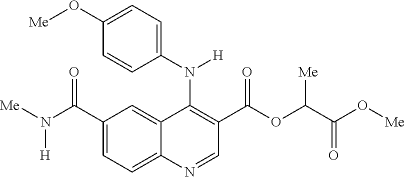

To a suspension of 4-[(4-methoxyphenyl)amino]-6-(methylcarbamoyl)-quinoline-3-carboxylic acid (300 mg, 0.85 mmol) and N,N-diisopropylethylamine (DIPEA, Hünig's base, 0.012 g, 0.09 mmol) in tetrahydrofuran (2 mL) at 0° C. was added L-methyl lactate (0.009 g, 0.08 mmol) and the reaction mixture was slowly brought to room temperature and stirred overnight. The reaction mixture was then concentrated in vacuo, extracted with ethyl acetate and purified on column (flash chromatography on silica gel, chloroform/methanol 9:1) to give 70 mg (19% yield) (2-methoxy-1-methyl-2-oxo-ethyl)-4-[(4-methoxyphenyl)amino]-6-(methyl-carbamoyl)quinoline-3-carboxylate. LC-MS (m/z) 437.8 (M+1). ¹H NMR (CDCl₃) δ 10.64 (s, 1H), 9.31 (s, 1H), 8.06 (m, 2H, aromatic), 7.79 (s, 1H), 7.17 (d, 2H, J=9 Hz), 6.98 (d, 2H, J=9 Hz), 5.50 (broad s, 1H), 5.54 (q, 1H), 3.87 (s, 3H), 3.83 (s, 3H), 2.87 (s, 3H), 1.73 (d, 3H, J=7 Hz).

### Example 7

### Acetoxymethyl 4-[(4-methoxyphenyl)amino]-6-(methylcarbamoyl)quinoline-3-carboxylate

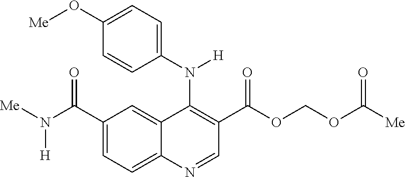

To a solution of 4-[(4-methoxyphenyl)amino]-6-(methylcarbamoyl)-quinoline-3-carboxylic acid (300 mg, 0.85 mmol) in N,N-dimethylformamid (5 mL) in a 10 mL microwave vial was added N,N-diisopropylethylamine (DIPEA, Hünig's base, 100 mg, 0.078 mmol) and acetic acid chloromethyl ester (10.6 mg, 0.85 mmol) under nitrogen atmosphere. This reaction mixture was irradiated at 150° C. for 30 minutes and the crude reaction mixture was poured over crushed ice. The reaction mixture was then extracted with ethyl acetate (50 mL), dried over anhydrous sodium sulfate and concentrated in vacuo. The residue was purified on column (flash chromatography on silica gel, chloroform: methanol over neutral alumina) to give 60 mg (16.5% yield) of acetoxymethyl 4-[(4-methoxyphenyl)amino]-6-(methylcarbamoyl)quinoline-3-carboxylate. LC-MS (m/z) 423.9 (M+1). ¹H NMR (CDCl₃) δ 11.43 (s, 1H,), 9.13 (s, 1H), 8.41 (d, 1H, J=8 Hz), 8.23 (d, 2H, J=8Hz), 8.10 (s, 1H), 7.25 (d, 2H, J=9 Hz), 7.05 (d, 2H, J=9 Hz), 6.05 (s, 2H), 3.90 (s, 3H), 2.93 (s, 3H), 2.19 (s, 3H).

### Example 8

### (Methylsulfonyl(phenyl)amino)methyl 4-[(4-methoxyphenyl)amino]-6-(methylcarbamoyl)quinoline-3-carboxylate

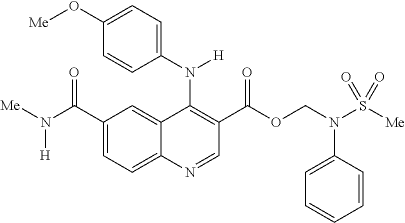

To a suspension of 4-[(4-methoxyphenyl)amino]-6-(methylcarbamoyl)-quinoline-3-carboxylic acid (CLT-28643) (0.1 g, 0.28 mmol) and N,N-diisopropylethylamine (DIPEA, Hünig's base, 0.1 g, 0.8 mmol) in tetrahydrofuran (5 mL) was stirred at 0° C. for 15 minutes. To this solution was added (2-chloro-1-methylsulfonylethyl)benzene (50 mg, 0.23 mmol), the reaction mixture was slowly brought to room temperature and stirred for 12 hours. The reaction mixture was concentrated in vacuo, extracted with ethyl acetate and purified on column (flash chromatography on silica gel, petroleum ether: ethyl acetate) to give 0.02 g (13% yield) of (methylsulfonyl(phenyl)amino)methyl 4-[(4-methoxyphenyl)amino]-6-(methylcarbamoyl)-quinoline-3-carboxylate. LC-MS (m/z) 534.7 (M+1). ¹H NMR (DMSO-d6) δ 9.90 (s, 1H), 8.97 (s, 1H), 8.54 (d, 1H, J=2 Hz), 8.46 (d, 1H, J=4.5 Hz), 8.11 (m, 1H), 7.95 (d, 1H, J=9 Hz), 7.50-7.43 (m, 5H), 7.04 (d, 2H, J=9Hz), 6.81 (d, 2H, J=9Hz), 5.60 (s, 2H), 3.71 (s, 3H), 3.21 (s, 3H), 2.76 (s, 3H).

### Example 9

### 2-[4-[(4-Methoxyphenyl)amino]-6-(methylcarbamoyl)quinoline-3-carbonyl]oxypropanoic Acid

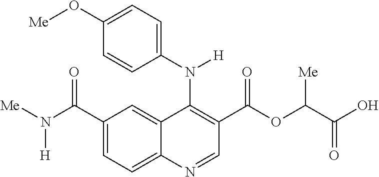

To a suspension of 4-[(4-methoxyphenyl)amino]-6-(methylcarbamoyl)quinoline-3-carboxylic acid (0.04 g, 0.09 mmol) in pyridine (15 mL) at room temperature was added lithium iodide (61 mg, 0.45 mmol) and the reaction mixture was refluxed at 110° C. for about 48 hours. The reaction mixture was diluted with hexane followed by acetonitrile. Subsequently, saturated ammonium chloride solution was added, the organic layer was separated and purified on column (preparative HPLC) to give 0.024 g (50% yield) of 2-[4-[(4-methoxyphenyl)amino]-6-(methylcarbamoyl)quinoline-3-carbonyl]oxypropanoic acid. LC-MS (m/z) 423.8 (M+1). ¹H-NMR (DMSO-d6) δ 10.08 (s, 1H), 8.96 (s, 1H), 8.55 (s, 1H), 8.49 (d, 2H, J=4.5 Hz), 8.25 (m, 1H), 7.95 (m, 1H), 7.10 (d, J=9 Hz), 6.91 (d, 2H, J=9 Hz), 4.87 (q, 1H, J=7 Hz), 3.79 (s, 3H), 2.70 (s, 3H), 1.44 (d, 3H, J=7Hz).

### Example 10

### 2-Imidazol-1-ylethyl 4-[(4-methoxyphenyl)amino]-6-(methylcarbamoyl)-quinoline-3-carboxylate

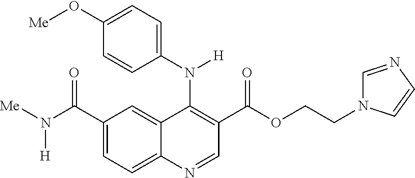

To a suspension of 4-[(4-methoxyphenyl)amino]-6-(methylcarbamoyl)-quinoline-3-carboxylic acid (1.0 g, 2.8 mmol) in dry tetrahydrofuran (15 mL) at 0° C. under nitrogen atmosphere was added 1-ethyl-3-(3-dimethylaminopropyl)-carbodiimide hydrochloride (2.0 g, 11.2 mmol), hydroxybenzotriazole (HOBt, 0.64 g, 4.84 mmol), triethylamine (3.2 mL, 2.3 mmol) and 2-hydroxyethylimidazole (0.65 g, 3.4 mmol). The reaction mixture was slowly brought to room temperature and stirred for 12 hours. The reaction mixture was concentrated in vacuo and after aqueous work up, extracted with dichloromethane, dried over anhydrous sodium sulfate and concentrated in vacuo. The residue was purified on column (flash chromatography on alumina gel, chloroform/methanol 99.8:0.2) to give 0.25 g (23% yield) of 2-imidazol-1-ylethyl 4-[(4-methoxyphenyl)amino]-6-(methylcarbamoyl)quinoline-3-carboxylate as a pale yellow solid. LC-MS (m/z) 446.2 (M+1). ¹H NMR δ (CDCl₃) 10.65 (s, 1H), 9.16 (s, 1H), 8.05 (dd, 1H, J₁=8.7 Hz, J₂=1.8 Hz), 7.97 (d, 1H, J=8.7 Hz), 7.84 (s, 1H), 7.76 (s, 1H), 7.22-7.10 (m, 3H), 7.08 (s, 1H), 6.97 (d, 2H, J=9.0 Hz), 5.67 (broad s, 1H), 4.75-4.60 (m, 2H), 4.50-4.35 (m, 2H), 3.87 (s, 3H), 2.88 (s, 3H).

### Example 11

### 2-Morpholinoethyl 4-[(4-methoxyphenyl)amino]-6-(methylcarbamoyl)-quinoline-3-carboxylate

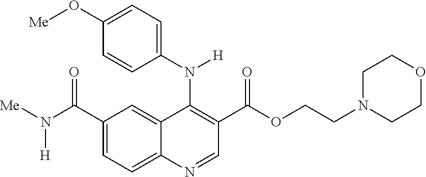

4-[(4-Methoxyphenyl)amino]-6-(methylcarbamoyl)-quinoline-3-carboxylic acid (1.0 g, 2.8 mmol) in N,N-dimethylformamid (15 mL) under nitrogen atmosphere was added N,N-diisopropylethylamine (DIPEA, Hünig's base, 4.1 mL, 2.48 mmol) and 4-(2-chloroethyl)-morpholinehydrochloride (1.0 g, 5.6 mmol), and the reaction mixture was irradiated in a microwave reactor at 120° C. for 30 minutes. After aqueous work up, the reaction mixture was extracted twice with dichloromethane, dried over anhydrous sodium sulfate and concentrated in vacuo. The residue was purified on column (flash chromatography on alumina gel chloroform/methanol 99.8:0.2) to give 0.22 g (16% yield) of 2-morpholinoethyl-4-[(4-methoxyphenyl)amino]-6-(methylcarbamoyl)-quinoline-3-carboxylate as a pale yellow solid. LC-MS (m/z) 465 (M+1). ¹H NMR (CDCl₃) δ 10.72 (s, 1H), 9.24 (s, 1H), 8.04 (dd, 1H, J₁=8.7 Hz, J₂=1.5 Hz), 7.98 (d, 1H, J=8.7 Hz), 7.81 (s, 1H, J=1.5 Hz), 7.15 (d, 21H, J=8.7 Hz), 6.95 (d, 2H, J=8.7 Hz), 5.52 (broad s, 1H), 4.60-4.50 (m, 2H), 3.87 (s, 3H), 3.80-3.75 (m, 4H), 2.90-2.80 (m, 5H), 2.70-2.55 (m, 4H).

### Example 12

### (5-Methyl-2-oxo-1,3-dioxol-4-yl)methyl 4-[(4-methoxyphenyl)amino]-6-(methylcarbamoyl)quinoline-3-carboxylate

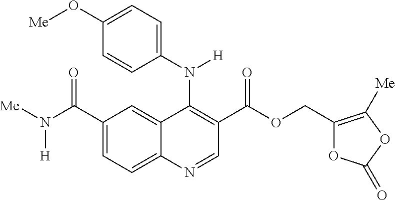

(a) Preparation of the intermediate 4-bromomethyl-5-methyl-2-oxo-1,3-dioxolene

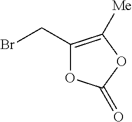

To a solution of 4,5-dimethyl-1,3-dioxol-2-one (342 mg, 3.0 mmol) in carbon tetrachloride (10 mL) was added azobisisobutyronitrile (AIBN, 9.8 mg, 0.06 mmol) and N-bromosuccinimide NBS (580 mg, 3.3 mmol). The reaction mixture was heated in the dark in a stem block at 78° C. for 20 minutes. The mixture was cooled and evaporated almost into dryness. The mixture was filtered and the residue was evaporated to give a light yellow solid, which contained 20% starting material Yield: 450 mg (58%). The mixture was used in the next step without further purification.

(b) Potassium carbonate (334 mg, 2.4 mmol) was added to a solution 4-[(4-methoxyphenyl)amino]-6-(methylcarbamoyl)-quinoline-3-carboxylic acid (0.17 g, 0.48 mmol) in N,N-dimethylformamide (5 mL) and the reaction mixture was stirred for 5 minutes. This mixture (solution) was added drop-wise to a solution of 4-bromomethyl-5-methyl-2-oxo-1,3-dioxolene (0.34 g, 1.74 mmol) in N,N-dimethylformamide (5 mL). The reaction mixture was stirred for 1 hour and concentrated in vacuo. The residue was partitioned between dichloromethane and aqueous saturated solution of sodium bicarbonate. The organic phase was dried over magnesium sulfate and concentrated in vacuo. The residue was purified on column (flash chromatography on silica gel, dichloromethane/methanol 95:5). The purest fractions from the chromathography were pooled and concentrated in vacuo, which gave (5-methyl-2-oxo-1,3-dioxol-4-yl)methyl 4-[(4-methoxyphenyl)amino]-6-(methylcarbamoyl)quinoline-3-carboxylate in 70% purity. This crude mixture was dissolved in dichloromethane and diethyl ether was added until formation of a yellow solid. The mixture was filtered and the yellow solid was washed twice with diethyl ether and dried in vacuo to give (5-methyl-2-oxo-1,3-dioxol-4-yl)methyl 4-[(4-methoxyphenyl)amino]-6-(methylcarbamoyl)quinoline-3-carboxylate with 95% purity (according to ¹H-NMR). Yield: 55 mg (25%). LC-MS (m/z) 463.9 (M+1). ¹H-NMR (CDCl₃) δ 10.67 (broad s, 1H), 9.19 (s, 1H), 8.05-7.99 (m, 2H), 7.82 (s, 1H), 7.17 (d, 2H, J=8.7 Hz), 6.98 (d, 2H J=8.9 Hz), 5.60 (broad s, 1H), 5.17 (s, 2H), 3.87 (s, 3H), 2.86 (d, 3H, J=5.1 Hz), 2.28 (s, 3H).

### Example 13

### 4-(4-Fluoro-phenylamino)-6-methylcarbamoyl-quinoline-3-carboxylic acid 2-imidazol-1-yl-ethylester

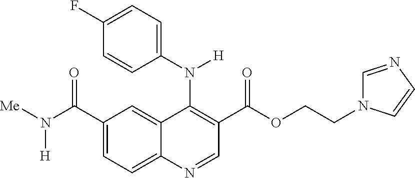

(a) Preparation of intermediary compound diethyl 2-((4-bromophenylamino)methylene)-malonate:

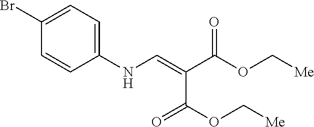

4-Bromoaniline (10 g) and diethoxymethylene malonate (12.6 g) were heated at 150° C. for 3 hours in a sealed tube. The reaction mixture was then cooled and diluted with n-hexane when the solid product precipitated out. This solid was filtered, washed several times with n-hexane and dried under vacuum to afford 17.8 g of 2-[(4-bromo-phenylamino)methylene]-malonic acid diethyl ester. ¹H NMR (300 MHz, CDCl₃) δ 11.03 (d, 1H, J=13 Hz, —NH—), 8.48 (d, 1H, J=13 Hz, —CH═C), 7.49 (m, 2H, aromatic), 7.10-7.01 (m, 2H, aromatic), 4.42-4.22 (m, 4H, —CH₂—CH₃), 1.45-1.26 (m, 6H, —CH₂—CH₃); LC-MS (m/z) 343.9 (M+1).

(b) Preparation of intermediary compound 6-bromo-4-chloroquinoline-3-carboxylic acid ethyl ester:

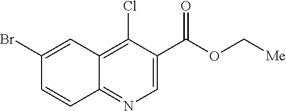

2-[(4-Bromophenylamino)methylene]malonic acid diethyl ester (5 g) was heated with POCl₃ (phosphoryl chloride, 31.5 mL) at 150° C. in a sealed tube for about 6 hours. The excess POCl₃ was removed in vacuo and the reaction mixture was diluted with dichloromethane. The dichloromethane extract was washed with aqueous sodium hydroxide solution (10%), dried over sodium sulphate and purified by column chromatography (Silica gel, hexane/ethyl acetate 80:20) to give 2.3 g of 6-bromo-4-chloroquinoline-3-carboxylic acid ethyl ester. ¹H NMR (300 MHz, CDCl₃) δ 9.22 (s, 1H, aromatic), 8.60 (d, 1H, J=2.1 Hz, aromatic), 8.04 (d, 1H, J=9 Hz, aromatic), 7.95-7.85 (m, 1H, aromatic), 4.53 (q, 2H, J=7 Hz, —CH₂—), 1.50 (t, 3H, J=7 Hz, —CH₃); LC-MS (m/z) 315.8 (M+1).

(c) Preparation of intermediary compound ethyl 6-bromo-4-(4-fluorophenylamino)-quinoline-3-carboxylate:

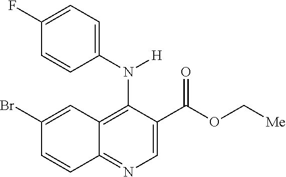

p-Fluoroaniline (0.106 g) and 6-bromo-4-chloroquinoline-3-carboxylic acid ethyl ester (0.3 g, 0.95 mmol) were mixed in dioxane and irradiated in a microwave reactor at 150° C. for 30 minutes. The reaction mixture was diluted with petroleum ether. The solid product obtained was filtered and dried to give 0.33 g of ethyl 6-bromo-4-(4-fluorophenyl-amino)quinoline-3-carboxylate. LC-MS (m/z) 389.4 (M+1).

(d) Preparation of intermediary compound ethyl 4-(4-fluorophenylamino)-6-(methylcarbamoyl)quinoline-3-carboxylate:

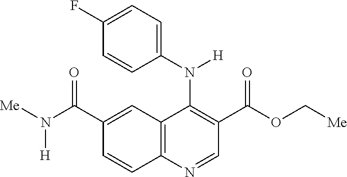

Ethyl 6-bromo-4-(4-fluorophenyl-amino)quinoline-3-carboxylate (0.3 g) was added to tetrahydrofuran followed by trans-di(μ-acetato)-bis[o-(di-o-tolylphosphino)-benzyl]dipalladium(II) (Herrmann's palladacycle, 0.038 mmol), tri tertiarybutyl phosphonium hexafluoborate) ([(t-Bu)₃PH]BF₄, 0.0385 mmol), molybdenum hexacarbonyl (Mo(CO)₆, 1.54 mmol), methylamine (4.6 mmol, 2N in tetrahydrofuran) and 1,8-diazabicyclo[5.4.0]undec-7-ene (DBU, 7.7 mmol). The reaction mixture was irradiated at 130° C. for 5 minutes in a microwave reactor. The reaction mixture was concentrated and then purified on column (silica gel, dichloromethane/methanol 98:2) to give 0.39 g of ethyl 4-(4-fluorophenylamino)-6-(methylcarbamoyl)quinoline-3-carboxylate as a solid. ¹H NMR (300 MHz, CDCl₃) δ 9.88 (s, 1H, —CONH—), 8.89 (s, 1H, aromatic), 8.72 (s, 1H, aromatic), 8.59 (d, 1H, J=4 Hz, aromatic), 8.15 (d, 1H, J=8.7 Hz, aromatic), 7.98 (d, 1H, J=8.7 Hz, aromatic), 7.16 (m, 4H, aromatic), 3.98 (q, 2H, J=7 Hz, —CH₂—), 2.80 (s, 3H, —NCH₃), 1.16 (t, 2H, J=7 Hz, —CH₃); LC-MS (m/z) 368.1 (M+1).

(e) Preparation of the intermediate compound 4-(4-fluorophenylamino)-6-(methyl-carbamoyl)quinoline-3-carboxylic acid

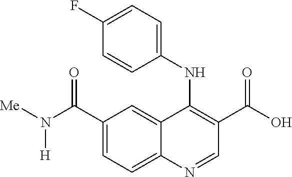

Ethyl 4-(4-fluorophenylamino)-6-(methylcarbamoyl)quinoline-3-carboxylate (0.03 g) was stirred with lithium hydroxide (0.128 g) in a mixture of 6 mL of methanol/tetrahydrofuran/water (2:2:2,) overnight. The reaction mixture was concentrated and the aqueous layer was washed with ethyl acetate. The aqueous layers were collected and acidified with aqueous hydrochloric acid and the precipitate formed was filtered and dried to give 0.022 g of 4-(4-fluorophenylamino)-6-(methylcarbamoyl)quinoline-3-carboxylic acid as a yellow solid. ¹H NMR (300 MHz, CD₃OD) δ 12.47 (bs, 1H, —C(O)OH), 9.12 (s, 1H, aromatic), 8.46 (s, 1H, aromatic), 8.23 (s, 1H, aromatic), 8.07 (d, 1H, J=8.4 Hz, aromatic), 7.92 (d, 1H, J=8.4 Hz, aromatic), 7.15 (m, 4H, aromatic), 2.17 (s, 3H, —NCH₃); LC-MS (m/z) 340.2 (M+1).

(f) To a solution of 4-(4-fluorophenylamino)-6-(methylcarbamoyl)quinoline-3-carboxylic acid (240 mg, 0.707 mmol) in a mixture of dichoromethane (6 mL), N,N-dimethylformamide (2 mL) and triethylamine (0.5 mL, 3.54 mmol) was added 1-ethyl-3-(3-dimethyllaminopropyl)-carbodiimide (EDC, 545 mg, 2.83 mmol) and hydroxybenzotriazole (HOBt, 58 mg, 0.42 mmol) at 0° C. The reaction mixture was stirred at the same temperature. After 20 minutes 1-hydroxymethyl imidazole (119 mg, 1.06 mmol) was added in one lot at 0° C. and continued the stirring for 24 hours at room temperature. The reaction mixture was quenched with water and extracted three times with dichloromethane (20 mL each time) and three times with a mixture of methanol and dichloromethane (10% methanol, 20 mL each time). The combined organic layer was dried over sodium sulfate, filtered and concentrated in vacuo. The crude product was washed with diisopropyl ether and recrystallized from dichloromethane to give 80 mg (26% yield) of 4-(4-fluorophenyl-amino)-6-methylcarbamoylquinoline-3-carboxylic acid 2-imidazol-1-yl-ethylester. ¹H-NMR (300 MHz, DMSO-d₆) δ 9.85 (bs, 1H), 8.87 (s, 1H), 8.61 (d, J=1.5 Hz, 1H), 8.54 (d, J=4.5 Hz, 1H), 8.15 (dd, J=8.7, 1.8 Hz, 1H), 7.96 (d, J=8.7 Hz, 1H), 7.68 (s, 1H), 7.21 (s, 1H), 7.14-7.07 (m, 4H), 6.90 (s, 1H), 4.26-4.20 (m, 4H), 2.78 (d, J=4.5 Hz, 3H). LC-MS (m/z, %): 419.8 (M+1, 91.9). HPLC: 94.7% purity.

### Example 14

### 4-(4-Fluoro-phenylamino)-6-methylcarbamoyl-quinoline-3-carboxylic acid imidazol-1-yl-methylester

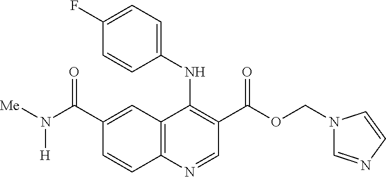

To a solution of (4-fluorophenylamino)-6-(methylcarbamoyl)quinoline-3-carboxylic acid (170 mg, 0.5 mmol) in a mixture of dichoromethane (5 mL), N,N-dimethylformamide (2 mL) and triethylamine (0.35 mL, 2.5 mmol) was added 1-ethyl-3-(3-dimethyllaminopropyl)-carbodiimide (EDC, 385 mg, 2.0 mmol) and hydroxybenzotriazole (HOBt, 41 mg, 0.303 mmol) at 0° C. The mixture was stirred at the same temperature. After 20 minutes 2-hydroxyethylimidazole (74 mg, 0.75 mmol) was added in one lot at 0° C. and stirred for 24 hours at room temperature. The reaction mixture was quenched with water and extracted three times with dichloromethane (20 mL each time) and three times with a mixture of methanol and dichloromethane (10% methanol, 20 mL each time). The combined organic layer was dried over sodium sulfate, filtered and concentrated in vacuo. The residue was washed with diisopropyl ether and recrystallized from dichloromethane to give 50 mg (24% yield) of 4-(4-fluoro-phenylamino)-6-methylcarbamoyl-quinoline-3-carboxylic acid imidazol-1-yl-methylester as a beige solid. ¹H-NMR (300 MHz, DMSO-d₆) δ 9.87 (bs, 1H), 8.91 (s, 1H), 8.62 (s, 1H), 8.52 (s, 1H), 8.13 (d, J=8.4 Hz, 1H), 7.96 (d, J=9.0 Hz, 1H), 7.80 (s, 1H), 7.27 (s, 1H), 7.14-7.10 (m, 4H), 5.95 (s, 2H), 2.79 (d, J=4.5 Hz, 3H). LC-MS (m/z, %): 433.7 (M+1, 94.8). HPLC: 95.3% purity.

### Example 15

### 2-Morpholinoethyl 4-[(4-fluorophenyl)amino]-6-(methylcarbamoyl)quinoline-3-carboxylate

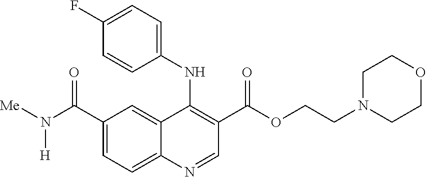

2-Chloroethylmorpholine hydrochloride (99 mg, 0.53 mmol) and N,N-diisopropylethylamine (DIPEA, Hünig's base, 38 mg, 0.29 mmol) was added to a solution of 4-[(4-fluorophenyl)amino]-6-(methylcarbamoyl)quinoline-3-carboxylic acid (100 mg, 0.29 mmol) in N,N-dimethylformamide (2 mL) The reagent mixture was heated under microwave conditions at 120° C. for 50 minutes. The reaction mixture was concentrated in vacuo and suspended in dichloromethane. An aqueous saturated solution of sodium hydrogen carbonate was added to the reaction mixture and extracted two times with dichloromethane. The combined organic phases were washed with an aqueous saturated solution of sodium hydrogen carbonate, dried over anhydrous magnesium sulfate and finally concentrated in vacuo. The residue was purified on column (silica gel, flash chromatography, dichloromethane/methanol 95:5) to give 25 mg (19% yield) of 2-morpholinoethyl 4-[(4-fluorophenyl)amino]-6-(methylcarbamoyl)-quinoline-3-carboxylate. LC-MS (m/z) 453.6 (M+1). ¹H-NMR (CDCl₃) δ 10.55 (s, 1H), 9.26 (s, 1H), 7.99 (s, 2H), 7.92 (s, 1H), 7.11-7.08 (m, 4H), 5.65 (d, 1H, J=4.3 Hz), 4.52 (triplet, 2H, J=11.3 Hz), 3.72-3.69 (m, 4H), 2.89 (d, 3H, J=4.7 Hz), 2.82 (t, 2H, J=11.7 Hz), 2.61-2.58 (m, 4H).

### Biological Assays

Cell Shape Assay

One of the used assays comprised a culture of PAE/VEGFR-2 and PAE/VEGFR3 cells. Morphological changes of the cells were recorded microscopically after addition of VEGF-A and VEGF-C respectively, followed by the test compound at a final concentration up to 100 μM. Growth inhibitions of the PAE/VEGFR-2 cells were detected in the presence of the compound of Example 1 according to the invention at 10 μM or lower. Furthermore, the inventive compounds were tested in PAE/VEGFR-3 cells and morphological changes of the cells were recorded microscopically after addition of the VEGF-C, followed by the test compound at a final concentration up to 100 μM. Growth inhibitions of the PAE/VEGFR-3 cells were detected in the presence of several of Examples according to the invention. The compounds were tested at 10, 50 and 100 μM. The effect of the test compounds in Table 1 is expressed as concentration of compound that inhibits the cell morphology induced by VEGF A and VEGF C. No effect means that no morphological changes were seen up to 100 μM compound concentrations.

Chemotaxis Assay

Additionally, the effect of the compounds was tested in this capacity of influencing chemotaxis. The test compounds were tested in porcine aorta endothelial (PAE) cells expressing VEGFR2 and VEGFR3 (PAE/VEGFR-2 and PAE/VEGFR-3). The method used is a modified Boyden chamber assay. The migration of the PAE cells expressing VEGFR2 and VEGFR3 receptors toward VEGF-A and VEGF-C respectively used as chemo-attractant was studied through micropore polycarbonate filter and was scored in the absence of serum. The assay was performed in the presence of compounds at 10 μM.

In Table 1, data from both the cell shape assay and the chemotaxis assay are shown. Thus, under “Cell Shape: PAE/VEGFR-2 with VEGF antagonist conc. (μM)” and “Cell Shape: PAE/VEGFR-3 with VEGF antagonist conc. (μM)” the concentration of the indicated inventive compound that gave restitution of cell morphology in the cell shape assay is shown. Data under “Chemotaxis: VEGFR-2% inhibition of cell migration” and “Chemotaxis: VEGFR-3% inhibition of cell migration” show the percentage inhibition of PAE cells expressing VEGFR-2 or 3 in the presence of 10 μM of the indicated inventive compound.

TABLE 1
Data from the cell shape assay and the chemotaxis assay

| | Cell Shape: | Cell Shape: | Chemotaxis: | Chemotaxis: |
|---|---|---|---|---|
| | PAE/VEGFR-2 | PAE/VEGFR-3 | VEGFR-2 | VEGFR-3 |
| | with VEGF | with VEGF | inhibition of cell | inhibition of cell |
| | antagonist conc. | antagonist conc. | migration (%) at | migration (%) at |
| | (μM) that gives | (μM) that gives | 10 μM compound | 10 μM compound |
| Example | inhibition | inhibition | concentration | concentration |
| 1 | 10 | Not tested | 52 | 53 |
| 2 | No effect | No effect | 39 | 45 |
| 3 | No effect | No effect | 28 | 36 |
| 4 | No effect | 100 | 21 | 63 |
| 5 | No effect | 100 | 2 | 64 |
| 6 | No effect | 50 | 23 | 69 |
| 7 | No effect | 50 | No effect | 25 |
| 8 | No effect | 100 | No effect | 18 |
| 9 | No effect | No effect | 38 | 52 |
| 10 | No effect | 10 | 32 | 53 |
| 11 | No effect | 10 | 10 | 83 |
| 12 | No effect | 100 | No effect | 46 |
| 13 | No effect | 50 | No effect | 27 |
| 14 | No effect | 50 | No effect | 30 |
| 15 | No effect | 100 | 31 | 59 |

Tumor Synograft Model

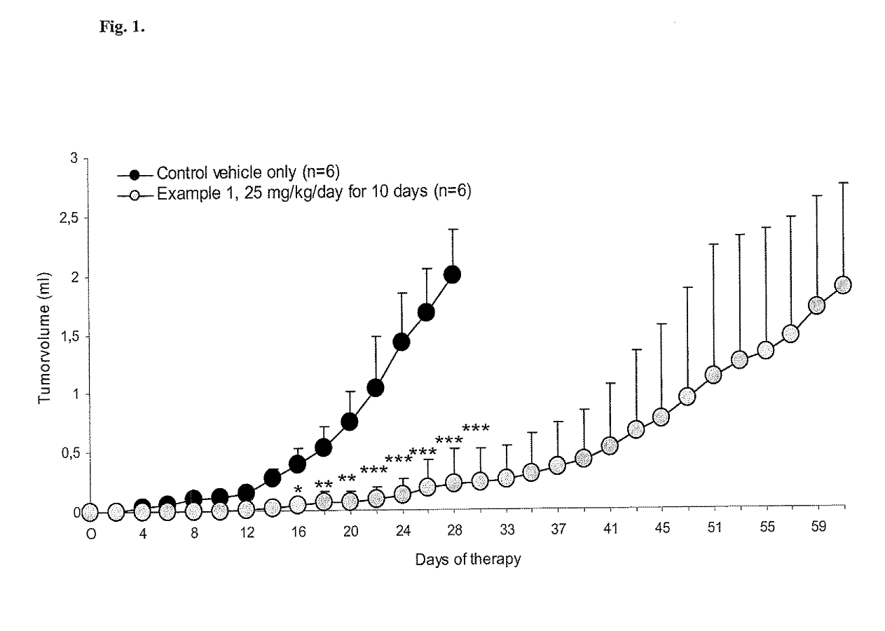

Female 6-week-old C57B1 mice were used for tumor studies. Approximately million human T241 wt mouse fibrosarcoma tumor cells growing in logarithmic phase were harvested and resuspended in media, and a single cell solution in a volume of 100 μL was implanted subcutaneously at the right flank of each animal. 6 Mice were used in the treated groups and 6 mice were used in the control groups. Systemic treatment by oral administration injections with either 50 μl of vehicle or the inventive compound (the compound of Example 1) (25 mg/kg/day) was begun at day at day 0 (zero). The inventive compound was administrated for 10 days. Visible tumors were present day 5-10 after implantation. Primary tumors were measured with digital calipers on the days indicated. Tumor volumes were calculated according to the formula: Length×width²×0.52 as reported. The compound of the invention showed convincing results for its effectiveness in this animal model (FIG. 1). It takes a significant number of days for the treated animals to reach the same tumor volume as the vehicle treated animals.

1. A compound of formula (I) 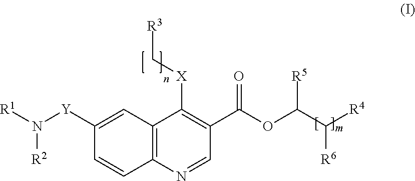
wherein:
n is 0 (zero) or 1;
m is 0 (zero), 1 or 2;
R¹ and R² are independently selected from hydrogen; branched or unbranched C₁-C₈ alkyl, C₂-C₈ alkenyl or C₂⁻C₈ alkynyl; monocyclic or bicyclic, saturated or unsaturated C₃-C₈ carbocyclyl; and monocyclic or bicyclic, saturated or unsaturated C₁-C₇ heterocyclyl wherein each heteroatom is independently selected from N, O and S; said alkyl, alkenyl, alkynyl, carbocyclyl or heterocyclyl optionally being substituted with 1, 2 or 3 groups R^{a};
R³ is selected from monocyclic or bicyclic C₆-C₁₀ aryl; and monocyclic or bicyclic C₁-C₉ heteroaryl or heterocyclyl, wherein in said heteroaryl and heterocyclyl each heteroatom is independently selected from N, O and S; said aryl, heteroaryl or heterocyclyl optionally being substituted with 1, 2, 3, 4 or 5 groups R^{b};
R⁴ is selected from —OC(O)R⁷; —C(O)OR⁷; —NR⁷R⁸; —C(O)NR⁷R⁸; monocyclic or bicyclic C₁-C₉ heteroaryl; and monocyclic or bicyclic, saturated or unsaturated C₁-C₉ heterocyclyl, wherein said heteroaryl and heterocyclyl optionally contains an oxo group in the ring, and wherein in said heteroaryl and heterocyclyl each heteroatom independently is selected from N, O and S; said heteroaryl and heterocyclyl optionally being substituted with 1, 2 or 3 groups R^{a};
R⁵ and R⁶ are independently selected from hydrogen; and branched or unbranched C₁-C₄ alkyl, C₂-C₄ alkenyl or C₂-C₄ alkynyl; said alkyl, alkenyl and alkynyl optionally being substituted with 1, 2, or 3 groups independently selected from fluorine and chlorine;
R⁷ is selected from hydrogen; and branched or unbranched C₁-C₄ alkyl, C₂-C₄ alkenyl or C₂-C₄ alkynyl; and phenyl; said alkyl, alkenyl, alkynyl and phenyl optionally being substituted with 1, 2, or 3 groups independently selected from fluorine and chlorine;
R⁸ is selected from hydrogen; branched or unbranched C₁-C₄ alkyl, C₂-C₄ alkenyl or C₂-C₄ alkynyl; monocyclic or bicyclic C₆-C₁₀ aryl; —S(O)₂R⁹; —C(O)OR⁹; and —C(O)R¹⁰; said alkyl, alkenyl, alkynyl or aryl optionally being substituted with 1, 2, or 3 halogen(s);
R⁹ is selected from hydrogen and branched or unbranched C₁-C₄ alkyl, C₂-C₄ alkenyl or C₂-C₄ alkynyl; said alkyl, alkenyl and alkynyl optionally being substituted with 1, 2, or 3 groups independently selected from fluorine and chlorine;
R¹⁰ is selected from hydrogen; branched or unbranched C₁-C₄ alkyl, C₂-C₄ alkenyl or C₂-C₄ alkynyl; and C₆ aryl; said aryl optionally being substituted with 1, 2 or 3 groups R^{a}; and said alkyl, alkenyl and alkynyl optionally being substituted with 1, 2, or 3 groups independently selected from fluorine and chlorine;
Y is selected from —C(O)—; —S(O)—; and —S(O)₂—;
X is selected from —NR^{c}—; —O—; and —S—;
each R^{a} is independently selected from halogen; hydroxy; carbonyl; methoxy; halomethoxy; dihalomethoxy; and trihalomethoxy;
each R^{b} is independently selected from halogen; carboxy; hydroxy; cyano; C₁-C₄ alkyl; C₂-C₄ alkenyl; C₂-C₄ alkynyl; C₁-C₄ alkyloxy; C₂-C₄ alkenyloxy; C₂-C₄ alkynyloxy;
C₁-C₄ alkylthio; C₂-C₄ alkenylthio; C₂-C₄ alkynylthio; C₁-C₄ alkyl; C₂-C₄ alkenyl or C₂-C₄ alkynyl secondary or tertiary amino; C₁-C₄ alkyl, C₂-C₄ alkenyl or C₂-C₄ alkynyl secondary or tertiary amido; C₁-C₄ alkyl, C₂-C₄ alkenyl or C₂⁻C₄ alkynyl carbonyl; C₁-C₄ alkyl, C₂-C₄ alkenyl or C₂-C₄ alkynyl sulfonyl; C₁-C₄ alkyl, C₂-C₄ alkenyl or C₂-C₄ alkynyl sulfonyloxy; C₁-C₄ alkyl, C₂-C₄ alkenyl or C₂-C₄ alkynyl secondary or tertiary sulphonamido; C₁-C₄ alkyl, C₂-C₄ alkenyl or C₂-C₄ alkynyl silyl; and C₁-C₄ alkyloxy, C₂-C₄ alkenyloxy, or C₂-C₄ alkynyloxy carbonyl; wherein any alkyl, alkenyl and alkynyl moiety optionally is substituted with 1, 2 or 3 groups independently selected from halogen, hydroxy, methoxy, halomethoxy, dihalomethoxy and trihalomethoxy; and
R^{c} is selected from hydrogen; and branched or unbranched C₁-C₄ alkyl, C₂-C₄ alkenyl or C₂-C₄ alkynyl;
wherein any Cₚ alkyl, alkynyl or alkenyl group having a number p≧4 of carbon atoms optionally includes a C_{q} carbocyclic portion of q of carbon atoms, whereby 3≦q<p;
or a pharmaceutically acceptable salt thereof.

2. A compound according to claim 1, wherein R¹ and R² are independently selected from hydrogen, C₁-C₄ alkyl, C₂-C₄ alkenyl and C₂-C₄ alkynyl, said alkyl, alkenyl, alkynyl, carbocyclyl or heterocyclyl optionally being substituted with 1, 2 or 3 groups R^{a}; R^{a} is halogen.

3. A compound according to claim 2, wherein R¹ represents hydrogen and R² represents C₁-C₄ alkyl.

4. A compound according to claim 1, wherein Y is —C(O)—.

5. A compound according to claim 1, wherein n is 0 (zero).

6. A compound according to claim 1, wherein R³ is phenyl, optionally substituted with 1, 2, 3, 4 or 5 groups R^{b}.

7. A compound according to claim 1, wherein R³ is phenyl, optionally substituted with 1 group R^{b}.

8. A compound according to claim 1, wherein X is —NR^{c}—.

9. A compound according to claim 8, wherein R^{c} is hydrogen.

10. A compound according to claim 1, wherein R⁴ is selected from —OC(O)R⁷; —C(O)OR⁷; —NR⁷R⁸; and —C(O)NR⁷R⁸.

11. A compound according to claim 1, wherein R⁷ is selected from C₁-C₄ alkyl and phenyl; R⁸ is selected from C₁-C₄ alkyl, —S(O)₂R⁹; —C(O)OR⁹ and —C(O)R¹⁰; R⁹ represents C₁-C₄ alkyl; and R¹⁰ represents phenyl.

12. A compound according to claim 1, wherein R⁴ is monocyclic or bicyclic C₁-C₉ heteroaryl or monocyclic or bicyclic, saturated or unsaturated C₁-C₉ heterocyclyl, wherein each heteroatom is independently selected from N, O and S.

13. A compound according to claim 1, wherein R⁴ is monocyclic C₁-C₄ heteroaryl; or monocyclic saturated or unsaturated C₁-C₄ heterocyclyl, wherein each heteroatom is independently selected from N, O and S.

14. A compound according to claim 13, wherein R⁴ is monocyclic C₁-C₄ heteroaryl, wherein each heteroatom is independently selected from N, O and S.

15. A compound according to claim 1, wherein each R^{b} is independently selected from C₁-C₄ alkyl, C₂-C₄ alkenyl or C₂⁻C₄ alkynyl, said alkyl, alkenyl and alkynyl, optionally being substituted with 1, 2 or 3 halogen(s).

16. A compound according to claim 1, wherein each R^{b} is independently selected from C₁-C₄ alkyloxy, C₂-C₄ alkenyloxy and C₂-C₄ alkynyloxy, said alkyloxy, alkenyloxy and alkynyloxy optionally being substituted with 1, 2 or 3 halogen(s).

17. A compound according to claim 1, wherein each R^{b} is selected from chloro, fluoro or trifluoromethyl.

18. A compound according to claim 1, wherein each R^{b} is selected from halogen.

19. A compound according to claim 1 which is:
(1H-imidazol-1-yl)methyl 4-(4-methoxyphenylamino)-6-(methylcarbamoyl)quinoline-3-carboxylate;
(methoxycarbonyl(methyl)amino)methyl 4-(4-methoxyphenylamino)-6-(methyl-carbamoyl)quinoline-3-carboxylate;
(N-methylbenzamido)methyl 4-(4-methoxyphenylamino)-6-(methylcarbamoyl)quinoline-3-carboxylate;
2-(dimethylamino)ethyl 4-(4-methoxyphenylamino)-6-(methylcarbamoyl)quinoline-3-carboxylate;
2-(dimethylamino)-2-oxoethyl 4-(4-methoxyphenylamino)-6-(methylcarbamoyl)quinoline-3-carboxylate;
(2-Methoxy-1-methyl-2-oxo-ethyl) 4-[(4-methoxyphenyl)amino]-6-(methylcarbamoyl)quinoline-3-carboxylate;
Acetoxymethyl 4-[(4-methoxyphenyl)amino]-6-(methylcarbamoyl)quinoline-3-carboxylate;
(Methylsulfonyl(phenyl)amino)methyl 4-[(4-methoxyphenyl)amino]-6-(methylcarbamoyl)quinoline-3-carboxylate;
2-[4-[(4-Methoxyphenyl)amino]-6-(methylcarbamoyl)quinoline-3-carbonyl]oxypropanoic acid;
2-Imidazol-1-ylethyl 4-[(4-methoxyphenyl)amino]-6-(methylcarbamoyl)-quinoline-3-carboxylate;
2-Morpholinoethyl 4-[(4-methoxyphenyl)amino]-6-(methylcarbamoyl)-quinoline-3-carboxylate;
(5-Methyl-2-oxo-1,3-dioxol-4-yl)methyl 4-[(4-methoxyphenyl)amino]-6-(methylcarbamoyl)quinoline-3-carboxylate;
4-(4-Fluoro-phenylamino)-6-methylcarbamoyl-quinoline-3-carboxylic acid 2-imidazol-1-yl-ethylester;
4-(4-Fluoro-phenylamino)-6-methylcarbamoyl-quinoline-3-carboxylic acid imidazol-1-yl-methylester;
2-Morpholinoethyl 4-[(4-fluorophenyl)amino]-6-(methylcarbamoyl)quinoline-3-carboxylate,
or a pharmaceutically acceptable salt thereof.

20. A compound according to claim 1, or a pharmaceutically acceptable salt thereof, for use in therapy.

21. A pharmaceutical composition comprising a therapeutically effective amount of a compound according to claim 1, or a pharmaceutically acceptable salt thereof, together with at least one pharmaceutically acceptable excipient.

22. A compound according to claim 1, or a pharmaceutically acceptable salt thereof, for use in the treatment of a disorder selected from cancer, diabetic retinopathy, age-related macular degeneration, inflammation, stroke, ischemic myocardium, atherosclerosis, macular edema and psoriasis.

23. (canceled)

24. A method of treating a mammal suffering from cancer, diabetic retinopathy, age-related macular degeneration, inflammation, stroke, ischemic myocardium, atherosclerosis, macular edema or psoriasis, comprising administering to said mammal in need thereof, a therapeutically effective amount of a compound according to claim 1 or a pharmaceutically acceptable salt thereof.
